(19) United States
(12) Patent Application Publication
(10) Pub. No.: US 2005/0261635 A1
(43) Pub. Date: Nov. 24, 2005

(54) Syringe

(75) Inventors: Phillip Owen Byrne, Newcastle Upon Tyne (GB); Penelope Rosemary Attridge, Middlesex (GB); Harry Raymond Ingham, Newcastle Upon Tyne (GB); John Polidoro, Manchester, CT (US); Spencer Kinsey, Manchester, CT (US); Chad Smutney, Manchester, CT (US)

Correspondence Address: NIXON & VANDERHYE, PC, 901 NORTH GLEBE ROAD, 11TH FLOOR, ARLINGTON, VA, 22203, US

(73) Assignee: BTG International Limited, London (GB)

(21) Appl. No.: 11/155,884

(22) Filed: Jun. 20, 2005

Related U.S. Application Data
(63) Continuation-in-part of application No. PCT/GB04/05357, filed on Dec. 20, 2004.

(30) Foreign Application Priority Data
Dec. 18, 2003 (GB) 0329269.5

(51) Int. Cl. A61M 5/00

(52) U.S. Cl. 604/208

(57) ABSTRACT

An auto-disable syringe is disclosed which prevents re-use of the syringe whetver dose is delivered on the first use. A restrictor bobbin comprises two elements: a plunger lock 1 and a barrel lock 20 which are relatively freely movable for a limited distance. The barrel lock includes a barb 27 which engages with the barrel interior wall to prevent proximal movement of the barrel lock, whilst the plunger lock includes a barb 6 which engages with the plunger to prevent distal movement of the plunger withy respect to the barrel lock. The plunger lock includes a further barb 9 which is engageable with the plunger to prevent proximal movement of the plunger. A lock release 40 inlcudes a latch tine 53 which, in the syringe as shipped, prevents engagement of the barb 9 with the plunger. After withdrawal of fluid, e.g. a liquid medicament, initial distal movement of the plunger causes the latch 53 to disengage with the barb 9, thereby preventing any further proximal movement of the plunger.

### TECHNICAL FIELD

The present invention relates to single use syringes, that is to say syringes which are intended to be used once and which are adapted in some way to prevent or at least to hinder further use.

### BACKGROUND ART

Disposable syringes are known in which a cylindrical barrel formed of transparent plastics material receives a piston which is slidable within the barrel. A shaft, which may be of cruciform or other, e.g. circular, section extends from the piston to a plunger handle for enabling the piston to be displaced along the barrel in a first or proximal direction to cause injectable fluid or body fluid to be drawn into the barrel via an aperture at one end of the barrel, or in a second or distal direction to cause the fluid to be expelled out of the aperture or to be injected into a patient via a needle.

Syringes of this type are generally sold as disposable items and are intended to be used only once to negate the risk of transmission of diseases between patients.

However, such syringes suffer from the drawback that it is difficult to prevent such syringes from being re-used, which re-use increases the risk of transmission of serious, life-threatening, conditions such as certain bacterial infections, viral hepatitis, and HIV.

Numerous designs have been proposed for syringes which are intended to negate or reduce the risk of the syringe being re-used. However, there are considerable challenges involed in designing a syringe which meets all desiderata, including, without limitation:
(a) allowing aspiration or flashback of blood to check for correct location of the needle in a patient;
(b) ability to deliver variable doses;
(c) smooth operation;
(d) simplicity of manufacture and use
(e) ability to inject diluent (e.g. sterile water) into a vial of powdered/lyophilised drug, and/or allow agitation of vial or syringe contents to assist powdered drug to go into solution.

EP0925083B1 discloses a single use syringe comprising a barrel with an internal annular groove at the proximal end and a further annular groove near the distal end but spaced from it. The plunger is formed with an integrally moulded barb-like flange adjacent the head, which flange bears resiliently against the barrel interior wall. The flange is able to move unrestricted in either proximal or distal direction along the majority of the barrel interior wall; however, the flange is only able to pass the annular grooves in the distal direction. The syringe is supplied with the plunger not fully depressed, so that the restrictor flange is on the proximal side of the more distal of the two grooves. Liquid may be drawn up into the syringe until the flange encounters the proximal groove, which prevents the plunger being withdrawn completely from the barrel. Liquid may be discharged freely from the syringe by depressing the plunger, the flange passing just beyond the more distal of the grooves when the plunger head is moved to the extreme distal end of the barrel. In this position, the plunger is now prevented from being withdrawn again because the flange will not pass the groove in the barrel. This design is elegant and simple but suffers from a number of drawbacks, not least the fact that there will inevitably be a slight jolt as the flange passes the more distal of the grooves when an injection is being given, and this jolt is likely to be felt by a patient. Furthermore, because of the unrestricted movement of the plunger between the two grooves, the syringe could potentially be used again and again provided the plunger is never fully depressed. After unpacking a syringe, it is normal to cycle the plunger over a short distance to check that the plunger is free to move and, if it is not, to free it: sometimes there can be a degree of adhesion between the plunger head and the barrel due to the length of time of storage, or due to the effects of gamma sterilisation. This is particularly the case with plunger heads which have had silicone lubricant applied to them. During this movement it would be relatively easy to lock the plunger of this syringe by moving the flange past the distal groove. This design is the only one of which the inventors are currently aware which properly can be used to aspirate a flash of blood for checking needle position in a patient prior to injecting a drug.

U.S. Pat. No. 5,000,737 discloses a syringe having a single piece metal barbed restrictor element located between the plunger shaft and the cylindrical interior syringe barrel wall. The element has barbs facing towards the plunger which prevent movement of the plunger distally with respect to the element, and barbs facing the barrel which prevent movement of the element proximally with respect to the barrel. The restrictor element is initially located near the proximal end of the barrel; thus initial proximal movement of the plunger to draw up liquid is permitted as the plunger can slide past the restrictor in this direction. Subsequent depression of the plunger to deliver liquid is permitted because the restrictor can move distally with respect to the barrel, and hence when the plunger is depressed it carries the element with it. Further movement is of course prevented. This design has many similarities with some of the embodiments set out below; however, it does not permit aspiration of a flash of blood nor repeated movement to assist in reconstitution of lyophilised drug.

U.S. 2003/0060759 discloses a design which has similarities to that of U.S. Pat. No. 5,000,737, but also some important differences. It, too, utilises a single piece metal barbed restrictor element mounted between the plunger shaft and the barrel interior wall, and it employes outwardly facing barbs to restrict the motion of the element with respect to the barrel. In this design, however, the plunger shaft has a stepped form with a shoulder part way along it. The restrictor element has a spring tang which acts against the barrel and forces it against the plunger shaft. The restrictor starts out at the proximal end of the shaft; withdrawal of the plunger past the restrictor is permitted until an enlarged diameter portion of the shaft, near the plunger head, comes into engagement with the restrictor. At the same time, the proximal end of the restrictor snaps behind the shoulder on the plunger; thereby movement of the plunger in either direction with respect to the restrictor is prohibited. The plunger can be depressed, carrying the restrictor with it to the distal end of the barrel, and then the plunger is incapable of further movement. This design is simple and has been used in a commercial vaccination syringe product. It suffers from the disadvantage that it may be used repeatedly, provided the user does not draw up the plunger to the point where the restrictor snaps into place on the reduced diameter part of the plunger shaft. Furthermore, once the restrictor has locked into place, which is of course the intention, aspiration of a flash of blood is not possible.

U.S. Pat. No. 5,222,942 discloses a design based on a ratchet system. A collar is installed in an initial distal position between plunger shaft and barrel. The plunger shaft is formed with annular ratchet teeth, and corresponding teeth are formed on the collar. The ratchet does not permit the plunger to be moved proximally past the collar, so when the plunger is initially drawn back in order to draw up liquid, it carries the collar with it to the extreme proximal end of the barrel where a formation on the barrel prevents the collar and plunger from being withdrawn completely from the barrel. The ratchet is such that the plunger may then be depressed past the collar to dispense liquid, and then of course the syringe is disabled. This design does not allow for aspiration of a flash of blood.

Definitions

Throughout this specification, the terms “distal” and “proximal” will be interpreted with respect to the user of the syringe, i.e. the person administering an injection. Thus the “proximal” end of the syringe is the open end into which the plunger is received, and the “distal” end is the nozzle/needle end.

The terms “usable length” and “usable extent” as used herein with respect to a syringe barrel means that portion of the barrel's length over which the plunger head is intended to be able to travel in the course of normal use, that is to say in the course of drawing up and discharging/injecting fluid. In some cases this can be a relatively small proportion of the overall length of the syringe, e.g. if it is desired that a restrictor bobbin be inserted deep into the barrel so as to make it harder for it to be removed by a user who may wish to deactivate the single use feature of the syringe.

The term “movement” as used herein, unless stated to the contrary, refers to movement substantially along the axis of the syringe, that is to say along the length of the syringe. Similarly the term “direction”, as used herein with regard to the movement of components, refers to one or the other direction along the axis of the syringe, i.e. the proximal sense or the distal sense.

The terms “restricted” and “restrict” as used herein with respect to movement of a component of the syringe with respect to another component are intended to mean that a degree of restriction of movment is provided which is appropriate for the particular syringe. What is important is that the overall design of the syringe is such that a user attempting to circumvent its non-reuse features is prevented from doing so or is at least severely hampered. Different degrees of “restriction” may be required for different designs. In modified versions of some of the embodiments described herein, the plunger may have a weak point and be designed to break if a user tries to move the plunger in a restricted direction, thereby rendering the syringe inoperable. In a syringe incorporating such a feature, the force needed to move syringe components in a “restricted” direction may not be very great, e.g. 30-100N, provided the plunger is designed to break when a force lower than this is applied. A syringe in which the plunger did not have such a weak point may require that a greater force is able to be resisted.

### SUMMARY OF THE INVENTION

According to a first aspect of the present invention, a syringe comprises:
(a) a barrel having a cylindrical interior surface substantially free of discontinuities over its usable extent;
(b) a plunger including a plunger head and a shaft;
(c) a restrictor bobbin movable with respect both to the barrel and the shaft to permit the drawing up and delivery of a fluid over the usable extent of the barrel, whilst limiting further use of the syringe;
(d) the restrictor bobbin and/or shaft together comprising one or more members which are freely movable with respect to each other over a limited distance which is smaller that the usable extent of the barrel so as to permit repeated distal and proximal cycles of movement of the plunger head over the said limited distance.

A syringe barrel which is substantially free of discontinuities over its usable extent helps to make for smooth operation of the plunger, especially during the delivery stroke when an injection is being given to a patient. Any jolts in the operation of the syringe during the delivery stroke are normally felt by the patient, and it is desirable to avoid this happening. Discontinuities in the inner cylindrical surface which would not be engaged by the sealing face of the plunger head (piston) in normal use or in use during the delivery stroke are not considered to be in the “usable extent” of the barrel. For example, a reduced diameter portion of the barrel may be provided at the far proximal end of the usable extent of the barrel for preventing complete withdrawal of the plunger from the barrel: this would not be considered part of the “usable extent”.

The provision of free relative movement of the restrictor bobbin and shaft over a limited predetermined extent allows repeated movement over a distance which is sufficiently small not to allow repeated injections with the syringe, or at least only to allow repeated injections of smaller quantities of fluid than the syringe would normally be capable of delivering. This degree of movement may be sufficient to allow for aspiration/flash-back of blood for checking needle location in a patient. Alternatively or in addition, this degree of movement may be sufficient for repeated movements to assist reconstitution of powdered/lyophilised drug into solution.

The said member(s) which are freely movable to permit repeated movement of the plunger head may be the restrictor bobbin in its entirety, or a part of it, or alternatively a part of the plunger shaft.

Optionally, the said movable member(s) may be provided by deformable portions of the bobbin or shaft, which are preferably resiliently deformable.

Preferably, said repeated movement is permitted at least when the plunger head is at or adjacent a proximal end of the said usable extent of the barrel.

It is preferable, though not essential, that the feature which allows aspiration of blood be operative at any position of the plunger so that the feature can be used whatever volume of injectable is contained in the syringe and also may be used to check the needle position in a patient prior to drawing a blood sample, i.e. when the syringe is substantially empty and the plunger in a distal position with respect to the barrel. Therefore, preferably the said limited repeated cycles of distal and proximal movement referred to above are permitted at substantially every relative position of the plunger and barrel over a the usable range. Furthermore, desirably the resistance to movement offered by the said freely movable members is such that proximally directed force on the plunger will move the said members in preference to any other means for moving the plunger with respect to the barrel. Repeated movements of the plunger over a small distance is thereby possible when the plunger is not at its most proximal position without inadvertently effecting further “permanent” retraction of the plunger.

The said free movement of the bobbin and shaft with respect to each other may be permitted in a first region of the shaft and resisted in a second region of the shaft.

The distance over which repeated movement is possible is, desirably, sufficient to aspirate a small volume of blood from a patient so as to check the position of the needle. All that is required for this function normally is that the aspirated blood be visible in the syringe. Factors which may need to be taken into account in determining the degree of movement required for this function may include, without limitation:
(a) the internal volume of the needle;
(b) if the needle is a separate entity, the internal volume of the syringe nozzle onto which the needle hub fits and any volume between the end of the nozzle and the internal base of the needle hub;
(c) any volume between the internal end of the syringe barrel and the plunger face when fully depressed;
(d) any “end float” of the plunger head: if the the plunger head is a separate entity from the shaft, then a certain amount of free play between the two is sometimes required to ensure the head “snaps on” to the shaft in manufacture;
(e) resilient deformation of the plunger head;
(f) the pressure drop which it is necessary to create to be sure that blood is aspirated;
(g) the volume of blood which needs to be present in the syringe barrel for the user to be able to discern its presence;
(h) a safety/error factor;
(i) the distance over which the user can discern movement easily: the “ergonomics” of the feature; and
(j) the diameter of the syringe barrel and plunger head.

The last of these points particularly will have a large effect on the volume as swept by the plunger head which corresponds to the distance over which the said repeated movement is possible.

The smallest possible volume for achieving this effect is about 10 microlitres: this might be the case e.g. if a short and thin (e.g. 1 cm, 30 gauge) needle is used which is moulded into the syringe barrel and if a plunger with an integrally moulded head is used, etc. In a 10 ml syringe this would correspond to 0.1% of the 10 ml swept volume of the syringe which equates to 0.1% of the usable length of the syringe. This is not precise since the usable volume of a syringe is often slightly more than its stated or graduated volume.

Normally a volume considerably greater than 10 microlitres would be required. For example, a large needle (e.g. 5 cm long, 18 gauge) may have an internal volume of approximately 50 microlitres. A standard luer nozzle has a dead space of about 50 microlitres and there will be additional dead space between the end of the nozzle and internal end of the needle hub. The plunger head of some syringes may have an end float of up to 1 mm which could correspond in a 10 or 20 ml syringe to 500 microlitres or more. In a 10 or 20 ml syringe a very small volume of blood may be more difficult to see in which case as much as 500 microlitres may be required. Adding these factors together with an allowance for error and for creating the pressure drop for withdrawing the blood might give a volume of as much as 2000 microlitres. In a 20 ml syringe this would correspond to 10% of the usable (graduated) swept volume of the syringe.

If it is desired that the syringe plunger should be capable of repeated movement of sufficient extent to agitate a drug powder and diluent mix, then it may be desirable to increase this range to as much as 50%. Of course, the larger the range of free movement, the greater the danger that this movement makes the syringe too easily re-usable for injecting drugs or other uses. The figure of 50% would probably be much too high for a 10 or 20 ml syringe, but for a very small syringe (0.5 ml or less) it may not be totally unreasonable from the point of view of preventing or at least hindering further use.

It can be seen that a large range of possibilities exist depending on the exact use to which the syringe is to be put. However, for most situations, a volume of between 50 and 500 microlitres would be preferable. 50 microlitres might be appropriate e.g. for a 5 ml syringe with a very small integral needle and where agitation of reconstituted drugs is not required. It would probably be desirable for this quantity to be more in the region of 100 or 150 or 200 microlitres, however, to allow a good margin for safety, human error, manufacturing tolerance, etc. 1000 microlitres might be appropriate for the same syringe where the ability to agitate reconstituted drugs is desirable, though this may still provide too great an opportunity for re-use of the syringe, and 500 microlitres may be more preferable.

Preferably, the distance over which the said repeated movement is possible is between 0.1% and 50% of the said barrel usable length, preferably between 1% and 20%. The lower end of this range might preferably be increased to 2%, 3% or 4% based on the example discussed above. The upper end of this range might preferably be reduced to 10% based on the example discussed above. However, these ranges should not be taken as limited to the particular syringe sizes discussed above which are presented by way of example only.

Accordingly, the said distance over which repeated movment is possible corresponds to a swept volume which is between 10 and 2,000 microlitres, preferably between 50 and 1000 microlitres, more preferably between 100 and 500 microlitres, or other absolute volume ranges corresponding to the percentage values of the total syringe volume mentioned above, for syringe total usable/measurable volumes of 0.5 ml, 1 ml, 1.5 ml, 2 ml, 2.5 ml, 3 ml, 5 ml, 10 ml and 20 ml.

The penultimate item in the above list of factors which may affect the desirable range of repeatable movement, factor (i), may require that a minimum distance is determined by what a user can practically work with. A reasonable range might be 0.5 mm to 20 mm, preferably 1 mm to 15 mm, more preferably 1.5 mm to 10 mm, still more preferably 2 mm to 7 mm or about 3 mm or 4 mm.

One or more of the said movable or deformable members which allow repeated movement of limited extent may incorporate or comprise a projection, tine, tang, barb, serration or other like formation or member in engagement with the barrel interior wall or the plunger to restrict motion of the said member(s) with respect to the barrel interior wall or plunger respectively in a predetermined direction (either proximal or distal).

The plunger may be provided with formations for restricting the movement of the restrictor bobbin with respect to the plunger shaft. One possibility is for the plunger shaft to be provided with a region, preferably defined by stop surfaces at each end of the said region which the bobbin cannot or cannot easily pass, over which region unrestricted repeated movement of the shaft with respect to the restrictor bobbin is possible.

The restrictor bobbin may take the form of a unitary member, which may have no slidable or substantially deformable parts, e.g. may be a unitary metal (e.g. pressed stainless steel) component. The bobbin may have one or more tines, barbs, serrations or the like formation(s) in engagement with the barrel interior wall so as to prevent movement of the bobbin with respect to the barrel in a predetermined direction, preferably whatever the position of the bobbin over the usable length of the barrel.

In addition to a first region as described above over which unrestricted movement of the shaft with respect to the bobbin is possible, the plunger shaft is preferably provided with a second region over which movement of the shaft with respect to the bobbin is restricted in one direction. The second region may be provided, for example, with a ratchet formation in which case the bobbin would be provided with a corresponding formation for engaging the ratchet formation on the plunger shaft, the formations acting together to restrict movement of the plunger with respect to the bobbin in one direction. Alternatively, the second region may be substantially smooth and a barb, serration, tine or similar may be provided on the bobbin facing the plunger and arranged to engage with the second region of the plunger so as to resist movement of the plunger with respect to the bobbin in a predetermined direction, but to permit proximal and distal movement of the plunger with respect to the bobbin over the said first region. The bobbin is preferably provided with a spring member acting against the barrel interior to bias the bobbin into engagement with the shaft at least when the bobbin is in registry with the said second region of the shaft.

The syringe would normally be supplied sterile packed with the plunger fully depressed and the bobbin in its starting position. One possibility if for the syringe to be provided pre-filled with diluent. In this event, a distal movement of the plunger is normally required to expel the diluent prior to a proximal movement to draw up reconstituted drug solution and the a further distal movement to deliver the drug solution. For this situation, it may be desirable to provide a restrictor member on the bobbin whose sense can be reversed, i.e. the direction in which it resists movement may be reversed. This could be achieved using barbs, tines or similar whose direction may be changed e.g. by applying a force to them via the plunger in the distal (depressed) position of the plunger in the barrel.

The syringe is preferably sterile packed with the plunger in a substantially fully depressed position.

Optionally, the syringe may be sterile packed with the plunger in a retracted position, especially if the syringe is supplied ready filled with sterile water or other diluent for reconstituting powedered or lyophilised drugs. In this case, it will be understood that a distal movement of the plunger is required to expel the diluent into a drug vial prior to withdrawing the plunger to draw up the dissolved drug and then expelling the drug solution.

In order to allow this, the invention encompasses the possibility that the bobbin may incorporate a restrictor member for restricting movement of the plunger with respect to the bobbin or the bobbin with respect to the barrel where the direction in which such movement is restricted is reversible.

The restrictor member in this case may comprise barbs or tines whose direction may be reversed by depressing the plunger when it is at or near its most distal position in the barrel.

Second, third and fourth aspects of the invention are set out below. The above preferable or optional features of the first aspect of the invention apply equally to the second, third and fourth aspects of the invention defined below. The above discussion of the range of possible distances and volumes which apply to the limited repeated cycling of the syringe plunger also applies equally to the second, third and fourth aspects.

According to a second aspect of the present invention, a syringe comprises:
(a) a plunger including a plunger head and a shaft;
(b) a barrel having a cylindrical interior surface substantially free of discontinuities over the usable range of movement of the plunger head;
(c) a restrictor bobbin adapted for unidirectional movement with respect to the barrel in a first direction and to the shaft in a second direction to permit the drawing up and delivery of a fluid over the said usable range, whilst limiting further use of the syringe;
(d) the restrictor bobbin and shaft or a part or parts of the bobbin or shaft being relatively movable freely over a predetermined limited distance, at least when the plunger is in a region adjacent the proximal end of the said usable range of movement, so as to permit repeated distal and proximal movement cycles of the plunger head over the said limited distance, the said limited distance being less than the said usable range of movement.

Preferably the said repeated distal and proximal movement cycles referred to above are permitted at substantially every relative position of the plunger and barrel over the said usable range.

Optionally, if a part of the restrictor bobbin or shaft is relatively movable (i.e movable with respect to another part of the bobbin or shaft respectively), then this part may be provided by or may include a deformable member, preferably a resiliently deformable member.

According to a third aspect of the present invention, a syringe comprises:
(a) a plunger including a plunger head and a shaft;
(b) a barrel having a cylindrical interior surface substantially free of discontinuities over the usable range of movement of the plunger head;
(c) a restrictor bobbin interengagable with and movable with respect to both the plunger and the shaft so as to restrict repeated cycles of distal and proximal movement of the plunger head other than over a predetermined limited range, the said predetermined limited range of repeated movement being available at least when the plunger is at or adjacent a proximal end of the said usable range of movement.

According to a fourth aspect of the present invention, a syringe comprises:
(a) a plunger including a plunger head and a shaft;
(b) a barrel having a cylindrical interior surface substantially free of discontinuities between proximal and distal ends of a full range of usable movement of the plunger head in the barrel;
(c) a restrictor bobbin located between the shaft and the barrel and having outer barbs, tines, serrations or the like interengagable with the barrel to restrict substantial movement of the said barbs, tines, serrations or the like with respect to the barrel in a predetermined direction;
(d) the restrictor bobbin and/or shaft carrying formations for limiting movement of the bobbin with respect to the plunger;
(e) the the plunger and bobbin being freely slidable with respect to each other over a limited range of movement, or the plunger or bobbin having relatively movable parts, whereby repeated proximal and distal cycles of plunger head movement with respect to the barrel are permitted over a predetermined limited range which is less than the said full usable range of movement of the plunger head;
(e) the said repeated cycles of movement being permitted at least when the plunger is at or adjacent the said proximal end of the of the usable range of movement of the plunger head.

The said formations carried by the restricor bobbin and/or shaft preferably comprise barbs, tines, serrations or the like carried by the restrictor bobbin and which are arranged to engage with the plunger shaft surface to limit movement in a predetermined direction.

According to a fifth aspect of the present invention, there is provided a syringe comprising:—
a barrel for containing fluid and having at least one aperture adjacent a first end thereof;
a piston having at least one shaft extending therefrom and adapted to be displaced in said barrel in a first direction from a first position to a second position to cause fluid to enter the barrel through at least one said aperture, and in a second direction from said second position to a third position to cause fluid to be expelled through at least one said aperture;
at least one restrictor bobbin mounted between said at least one shaft and said barrel for sliding movement relative to said barrel and the corresponding shaft;
at least one first gripping member acting between a respective said restrictor bobbin and a respective said shaft for sliding movement relative to said shaft, wherein at least one said first gripping member has a greater resistance to sliding movement relative to the corresponding shaft in said first direction than in said second direction, such that movement of said piston from said second position to said third position causes at least one said restrictor bobbin to move along said barrel in said second direction; and
at least one second gripping member acting between a respective said restrictor bobbin and said barrel to cause the corresponding said restrictor bobbin to have a greater resistance to sliding movement relative to the barrel in said first direction than in said second direction, such that movement of the piston from said third position to said second position, subsequently to movement of said piston from said second position to said third position, without damaging said syringe, is prevented.

By providing a syringe in which movement of the piston from said third position to said second position subsequently to movement of said piston from said second position to said third position is prevented, this provides the advantage of preventing refilling of the syringe after the piston has been displaced from the second position to the third position to expel fluid out of the barrel. In other words, the syringe is prevented from being re-used after it has been used to administer an injection or remove bodily fluids, as a result of which the risk of transmission of disease is significantly reduced.

In a preferred embodiment, the first position is substantially coincident with the third position.

At least one said second gripping device may be adapted to engage said barrel such that movement of the corresponding said restrictor bobbin in said first direction relative to the barrel causes damage to the surface of said barrel to prevent said piston subsequently forming a fluid seal with said barrel.

This provides the advantage that forced withdrawal of the piston subsequently to movement of the piston from the second to the third position causes damage to the smooth walls of the barrel, thereby destroying the integrity of the fluid seal between the piston and the barrel. As a result, the syringe can no longer generate the necessary suction to be filled with fluid or pressure to expel fluid. This in turn makes re-use of the syringe more difficult.

At least one said first gripping member and the remaining part of the restrictor bobbin may be adapted to cooperate to allow limited sliding movement of said piston relative to the barrel in said first direction subsequently to movement of said piston from said second position to said third position.

This provides the advantage of enabling slight withdrawal of the piston during use to determine whether a needle connected to the syringe has been inserted into a blood vessel. For example, an intramuscular injection, to be injected into muscle tissue, should not be injected into a blood vessel, and slight withdrawal of the piston causes a visible amount of blood to be drawn into the barrel if the needle of the syringe has punctured a blood vessel. However, this safety feature will not permit a significant amount of injectable material to be subsequently withdrawn into the syringe after the primary injection has occurred. The discussion above regarding the degree to which this slight withdrawal is permitted, in terms of distance of movment of the plunger or aspirated volume, applies here.

At least one first gripping member and the corresponding restrictor bobbin may surround the corresponding shaft, wherein the gripping member is adapted to move relative to the corresponding restrictor bobbin between first and second stop positions.

At least one said first and/or second gripping member may comprise at least one metal tine.

At least one first and/or second gripping member may comprise elastomeric material.

An inner wall of the barrel may comprise a first plastics material, and at least one second gripping member may comprise a second plastics material harder than said first material.

An outer wall of at least one said shaft may comprise a third plastics material, and at least one corresponding said first gripping member may comprise a fourth plastics material harder than said third material.

In a sixth aspect of the invention, a syringe comprises:
(a) a plunger including a plunger head and a shaft;
(b) a barrel;
(c) a restrictor bobbin located between the shaft and the barrel and having an outer barb, tine, serration or the like engaged with the barrel and oriented so as to limit movement of the bobbin with respect to the barrel in a first direction;
(d) the restrictor bobbin and/or shaft carrying a formation for limiting movement of the bobbin with respect to the plunger in the said first direction;
(e) the bobbin being provided with a further barb, tine, serration or other formation oriented or arranged so as to restrict movement of the bobbin with respect to one of the barrel and plunger shaft in a second direction opposed to the first direction;
(f) a latch member for disabling the said restricting function of the further barb, tine, serration or other formation until a predetermined point in an operation cycle of the syringe.

The said predetermined point in the operation cycle is preferably initial movement of the plunger in the distal direction. With such a syringe, a dose of medicament may be drawn up by moving the plunger proximally as normal. Then, in order to deliver the medicament, the plunger is moved distally; as soon as the plunger is moved a small distance distally, the latch referred to above is disengaged rendering further substantial proximal movement of the plunger impossible. This renders the syringe unusable for any purpose except delivery of the dose of medicament which has already been drawn up, and this is true irrespective of whether the dose fills all or only a part of the usable volume of the syringe.

It will be appreciated that the prevention of movement of the plunger in the proximal direction means that drawing up further liquid with the syringe is essentially prevented. However, it is contemplated that a degree of unlimited repeatable movement of the plunger in the proximal and distal directions over a small defined distance may be permitted, as described in detail above in connection with the first to fifth aspects of the invention. In fact, this is preferred, and the embodiments described below incorporate this feature.

In a preferred form, the barrel has a cylindrical interior surface substantially free of discontinuities between proximal and distal ends of a full range of usable movement of the plunger head in the barrel.

The said formation carried by the restrictor bobbin and/or shaft for limiting movement of the bobbin in the first direction preferably comprises a barb, tine, serration or the like carried by the restrictor bobbin and which is arranged to engage with the plunger shaft surface.

Preferably, the plunger and bobbin are freely slidable with respect to each other over a limited range of movement, or the plunger or bobbin have relatively movable parts, whereby repeated proximal and distal cycles of plunger head movement with respect to the barrel are permitted over a predetermined limited range which is less than the said full usable range of movement of the plunger head. If this feature is provided, it is preferred that the said repeated cycles of movement are permitted at least when the plunger is at or adjacent the said proximal end of the usable range of movement of the plunger head, and preferably over the full usable range of the syringe.

The said latch member, which is disengageable at a predetermined point in the operation cycle of the syringe, e.g. by an intial distal movement of the plunger, may take the form of a separate member secured to the barrel, preferably at or adjacent the proximal end of the usable range of the barrel, which in an engaged state extends between the plunger shaft and a portion of the restrictor bobbin. For example, the member may comprise a projection extending distally between the plunger shaft surface and a barb, tang, serration etc on the restrictor bobbin; initial distal movement of the plunger may cause the barb to slide off the latch member as the bobbin moves distally together with the plunger.

Alternatively, the said latch member may be provided as a separate member on or adjacent the proximal side of the plunger head, which in an engaged state extends between the barrel interior wall and a portion of the restrictor bobbin. Optionally the member may be an integral part of the plunger head. For example, the latch member may comprise a projection extending proximally from the plunger head, between the barrel and a barb on the restrictor bobbin. When the plunger is drawn back (proximally), the member remains between the barrel wall and the barb, but as soon as the plunger is moved distally, the barb slides off the latch member and engages with the barrel wall.

The restrictor bobbin may be made in two relatively movable parts or a single part with a first region which may be deformed with respect to a second. In this event the latch member may be provided on one of the two parts or regions. For example, it may comprise a projection extending from one of the parts or regions of the restrictor bobbin and, in the engaged state, between the plunger or barrel surface and a barb, tine, serration (or the like) on the other part of the bobbin.

The latch member is preferably made from a material which is harder than the material of the syringe barrel and plunger shaft, such that the said barb, tine, serration, etc may not penetrate the latch member material. Preferably, the latch member material is such that the barb, tine, serration etc will slide over it relatively freely, preferably even when the barb, tine serration, etc is biased against the latch member. The latch member is preferably metal, more preferably stainless steel. The barb, tine, serration, etc is preferably metal, e.g. stainless steel. The syringe plunger and/or barrel are preferably of plastics material, e.g. polypropylene.

A number of embodiments of the invention will now be described, by way of example only and not in any limitative sense, with reference to the accompanying drawings, in which:—

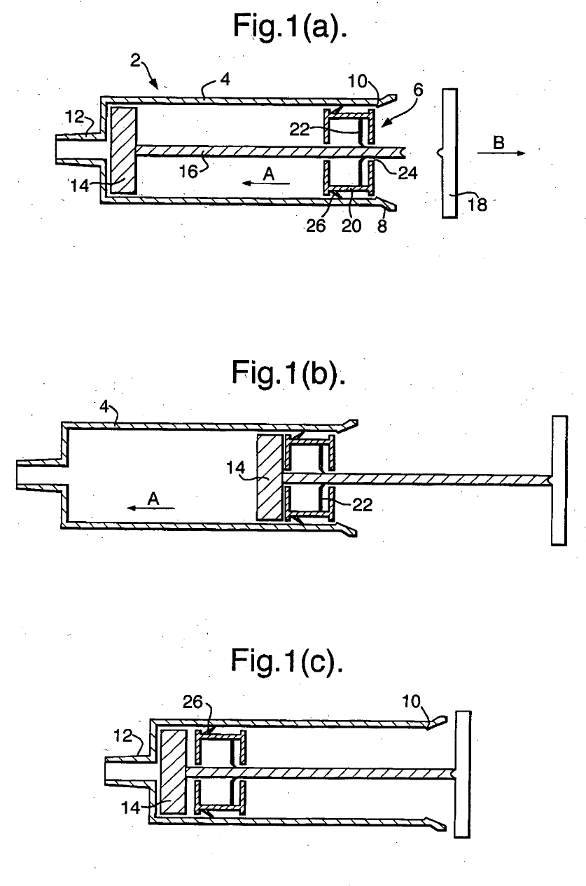

FIG. 1a is a schematic cross-sectional side view of a first embodiment of syringe according to the present invention in an initial manufacturer's packaging position;

FIG. 1b is a view, corresponding to FIG. 1a, of the syringe of FIG. 1a with the piston thereof withdrawn to enable filling of the barrel;

FIG. 1c is a view, corresponding to FIG. 1a, of the syringe of FIG. 1a with the piston thereof depressed to eject liquid from the barrel;

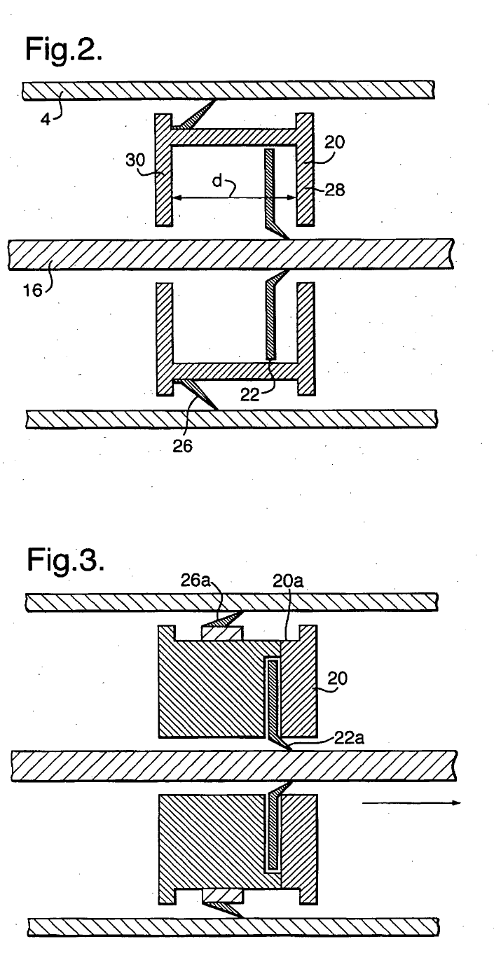

FIG. 2 is a schematic cross-sectional side view of the restrictor bobbin of the syringe of FIG. 1 in the positions shown in FIGS. 1a and 1b;

FIG. 3 is a view similar to FIG. 2 of the restrictor bobbin of a modified version of the first embodiment;

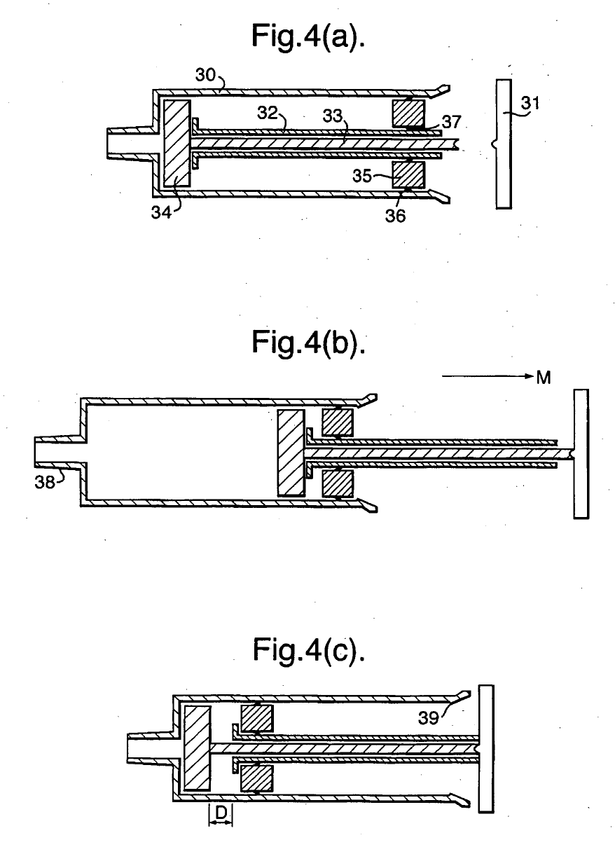

FIG. 4a is a schematic cross-sectional side view of a second embodiment of syringe according to the present invention in an initial manufacturer's packaging position;

FIG. 4b is a view, corresponding to FIG. 4a, of the syringe of FIG. 4a with the piston thereof withdrawn to enable filling of the barrel;

FIG. 4c is a view, corresponding to FIG. 4a, of the syringe of FIG. 4a with the piston thereof depressed to eject liquid from the barrel;

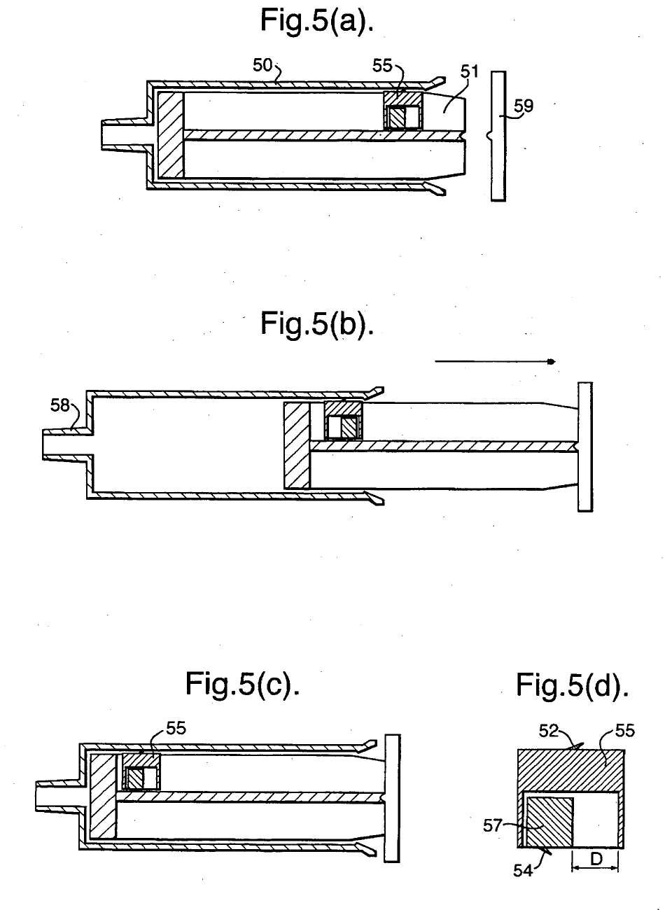

FIG. 5a is a schematic and partly exploded cross-sectional side view of a third embodiment of syringe according to the present invention in an initial manufacturer's packaging position;

FIG. 5b is a view, approximately corresponding to FIG. 5a, of the syringe of FIG. 5a with the piston thereof withdrawn to enable filling of the barrel;

FIG. 5c is a view, approximately corresponding to FIG. 5a, of the syringe of FIG. 5a with the piston thereof depressed to eject liquid from the barrel;

FIG. 5d is a schematic side cross-section of the restrictor bobbin of the third embodiment;

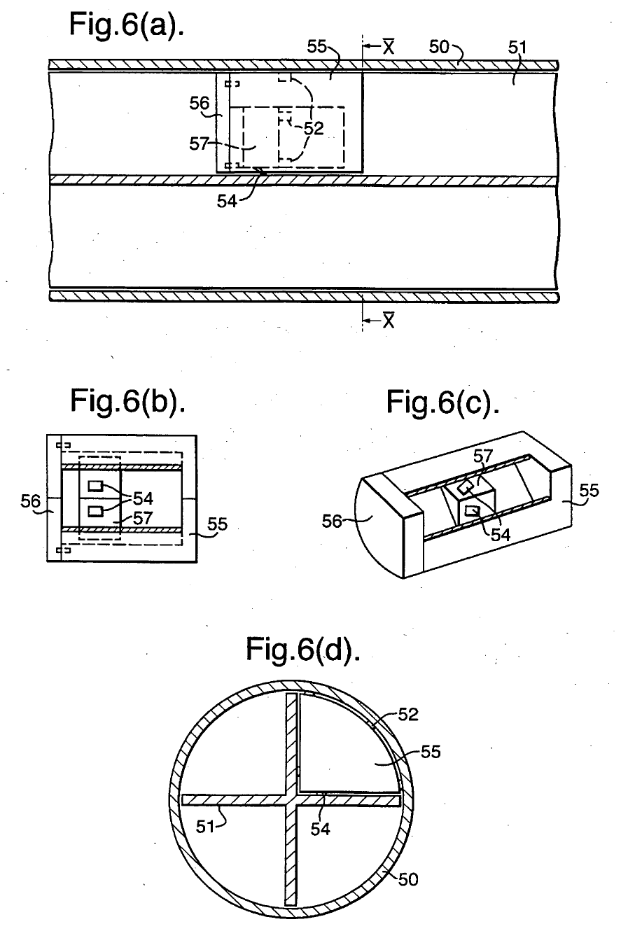

FIG. 6a is a schematic scrap sectional view from the side of part of the third embodiment;

FIG. 6b is a schematic side view of the restrictor bobbin of the third embodiment;

FIG. 6c is a schematic perspective view of the restrictor bobbin of the third embodiment;

FIG. 6d is a schematic sectional view taken on the line X-X in FIG. 6a;

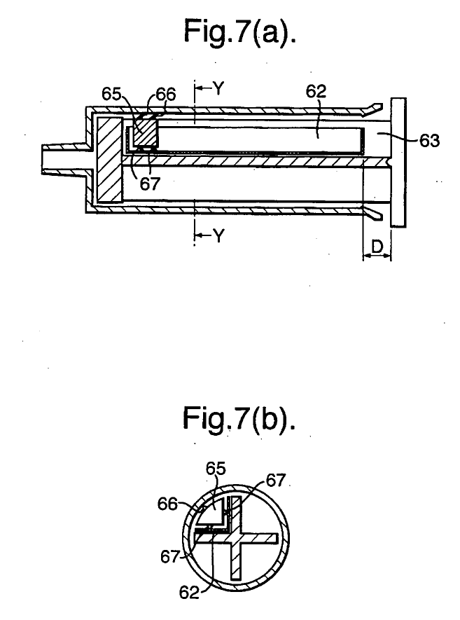

FIG. 7a is a schematic cross-sectional side view of a fourth embodiment of syringe according to the present invention in an initial manufacturer's packaging position;

FIG. 7b is a view, corresponding to FIG. 7a, of the syringe of FIG. 7a with the piston thereof withdrawn to enable filling of the barrel;

FIG. 7c is a view, corresponding to FIG. 7a, of the syringe of FIG. 7a with the piston thereof depressed to eject liquid from the barrel;

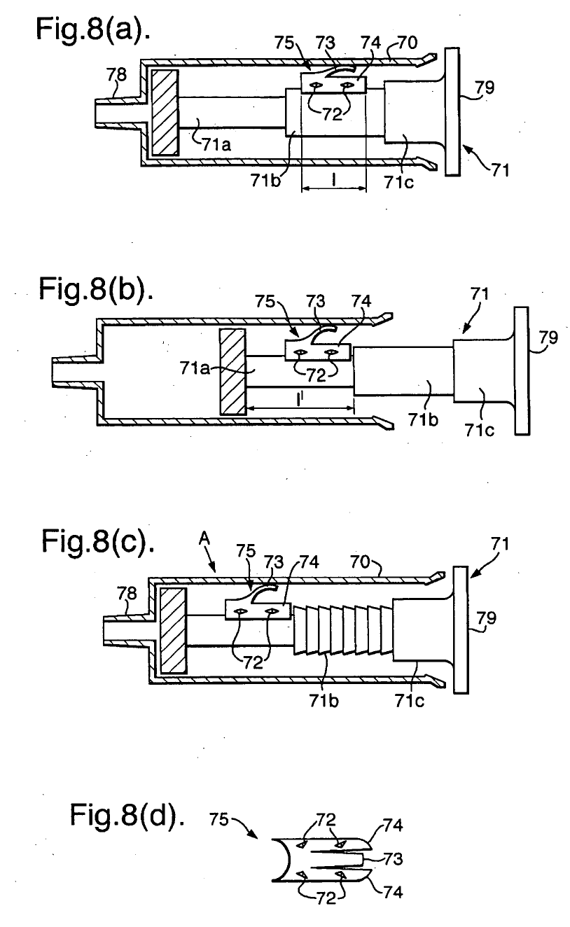

FIG. 8a is a schematic cross-sectional side view of a fifth embodiment of syringe according to the present invention in an initial manufacturer's packaging position;

FIG. 8b is a view, corresponding to FIG. 8a, of the syringe of FIG. 8a with the piston thereof withdrawn to enable filling of the barrel;

FIG. 8c is a view, corresponding to FIG. 8a, of the syringe of FIG. 8a with the piston thereof depressed to eject liquid from the barrel;

FIG. 8d is a view in the direction A in FIG. 8c of the restrictor bobbin of the fifth embodiment;

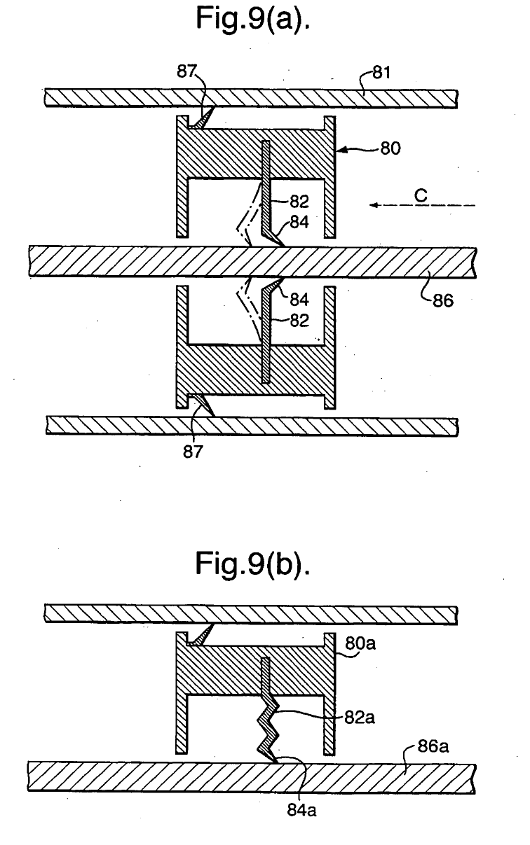

FIG. 9a is a scrap sectional view similar to FIG. 2 of a sixth embodiment;

FIG. 9b is a view similar to FIG. 9a but of only half the diameter of the syringe, showing a modification of the sixth embodiment;

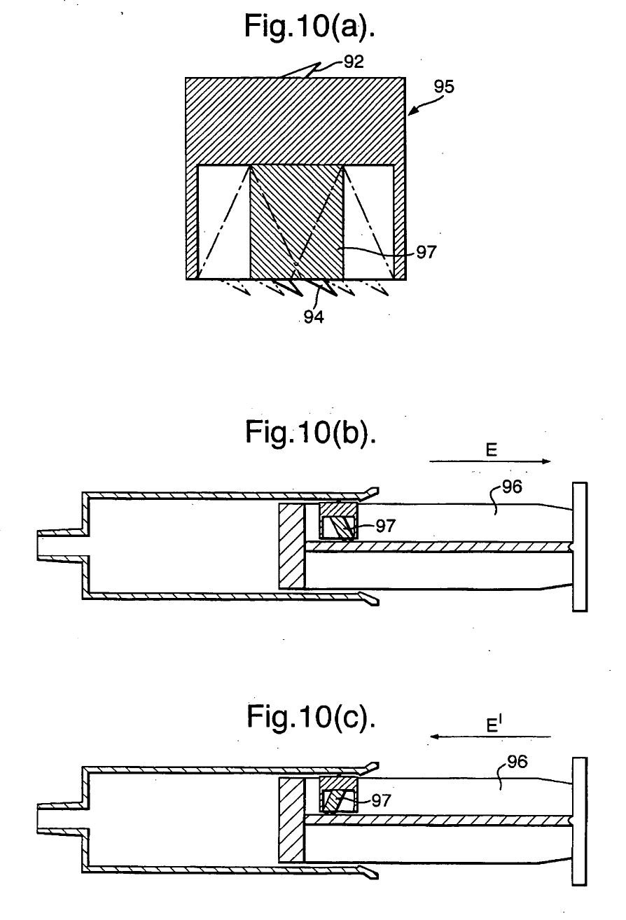

FIG. 10a is a sectional view similar to FIG. 5d of the restrictor bobbin of a seventh embodiment;

FIG. 10b is a side sectional view similar to FIG. 5b of the entirety of the seventh embodiment, showing force being applied to the plunger in a proximal direction;

FIG. 10c is a view similar to FIG. 10b showing force being applied to the plunger in a distal direction;

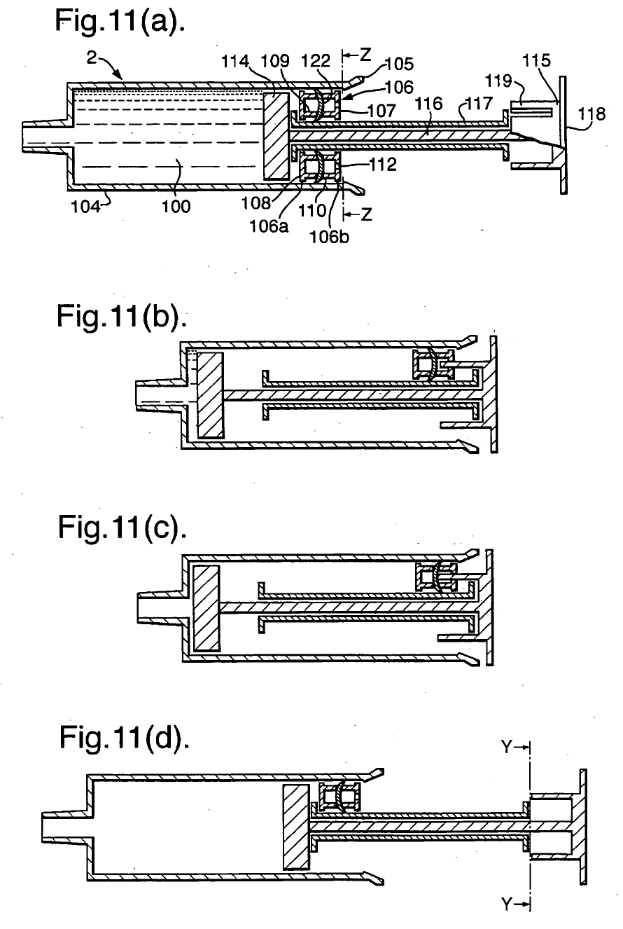

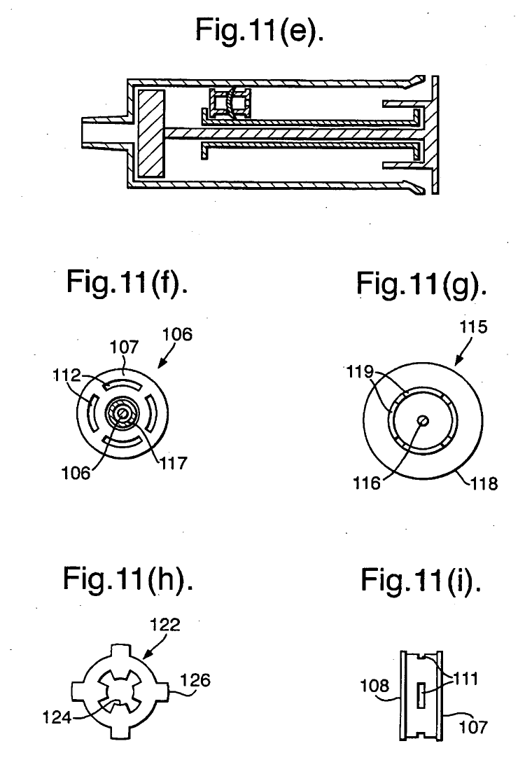

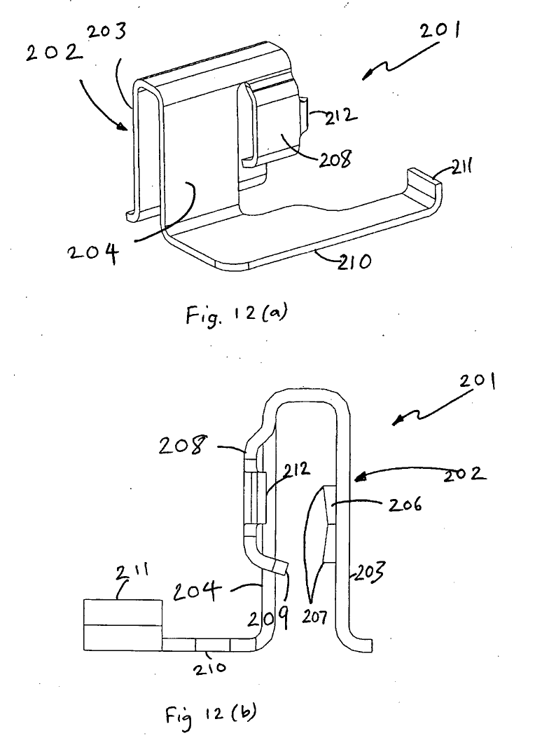

FIG. 11a is a side sectional view of an eighth embodiment as supplied from the manufacturer, charged with diluent liquid;

FIG. 11b is a similar view of the syringe of FIG. 11a, only partly showing the restrictor bobbin, with the plunger almost completely depressed to expel diluent;

FIG. 11c is a similar view to FIG. 11b with the plunger completely depressed to expel diluent;

FIG. 11d is a similar view to FIG. 11b with the plunger retracted having drawn up an injectable liquid;

FIG. 11e is a similar view to FIG. 11b with the plunger depressed after expulsion of injectable liquid;

FIG. 11f is a view along the line Z-Z in FIG. 11a of the plunger shaft (in section) and the restrictor element of the syringe of FIGS. 11a-e;

FIG. 11g is a view along the line Y-Y in FIG. 11d of the proximal end of the plunger of the syringe of FIGS. 11a-e;

FIG. 11h is an axial view of the spring washer shown fully in FIG. 11a and in part in FIGS. 11b-e; and

FIG. 11i is a side view of a part of the restrictor bobbin of the syringe of FIGS. 11a-e;

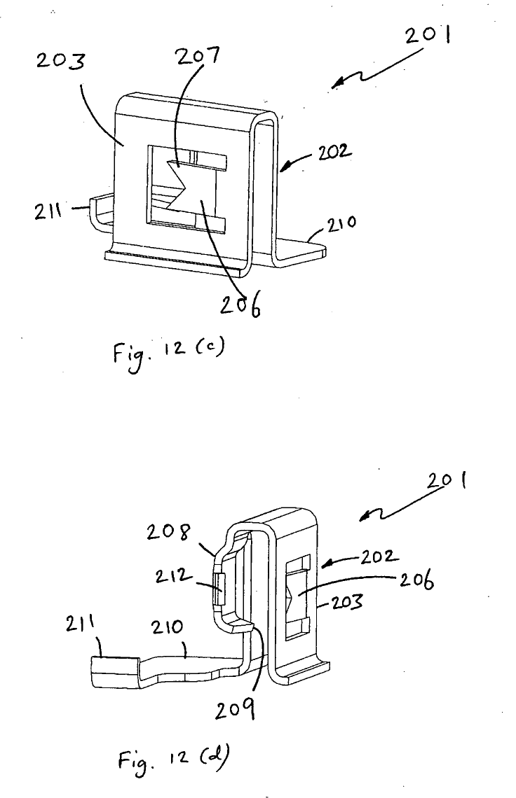

FIG. 12a is a perspective view in a first direction of a plunger lock component of a ninth embodiment of the invention;

FIG. 12b is an elevation of the component of FIG. 12a;

FIG. 12c is a perspective view in a second direction of the component of FIG. 12a;

FIG. 12d is a perspective view in a third direction of the component of FIG. 12a;

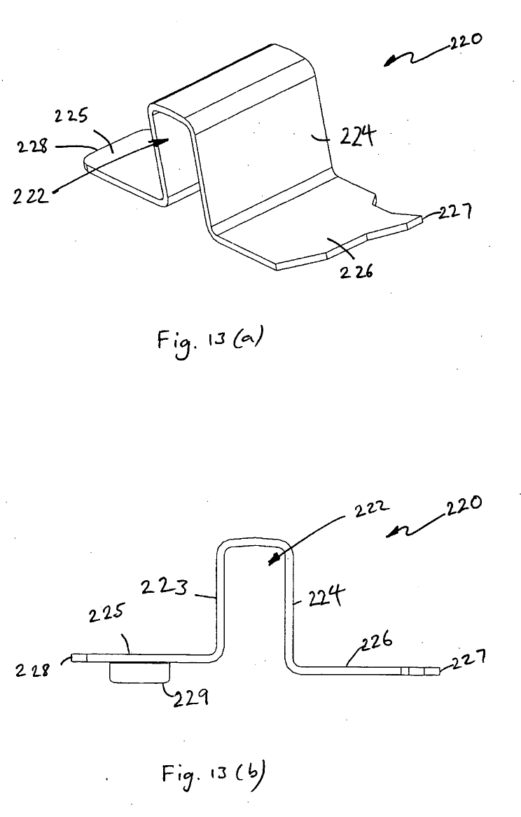

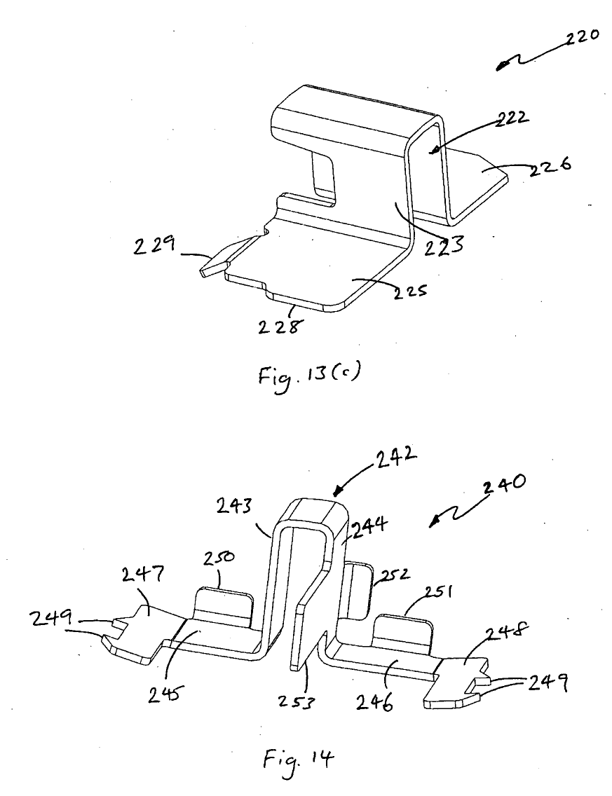

FIG. 13a is a perspective view of a barrel lock component of the ninth embodiment;

FIG. 13b is an elevation of the component of FIG. 13a;

FIG. 13c is a perspective view from a different direction of the component of FIG. 13a;

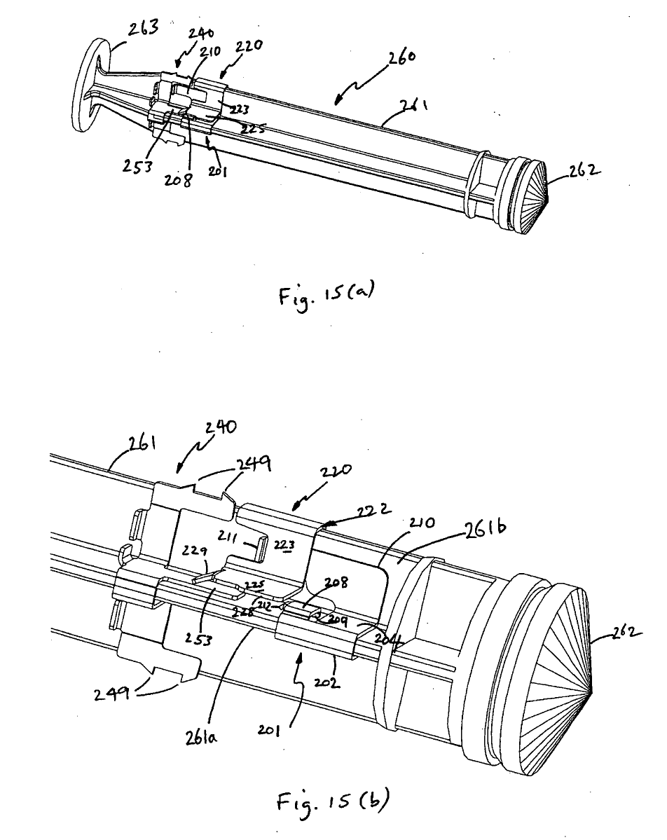

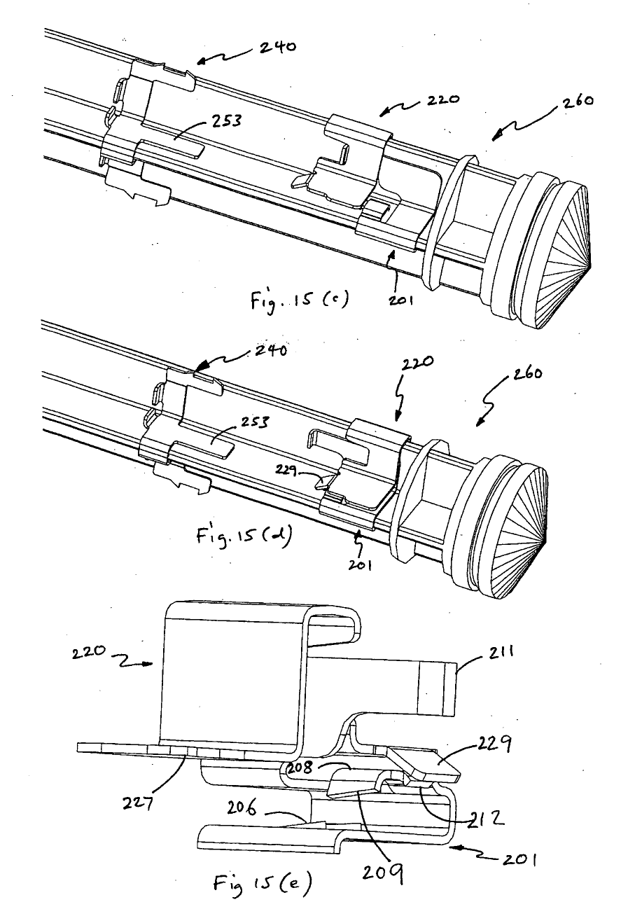

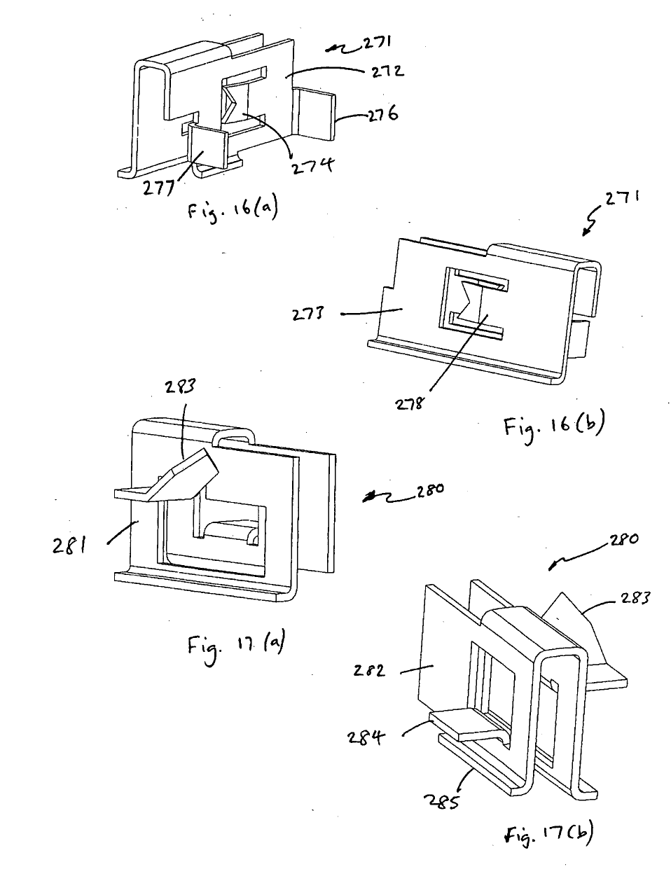

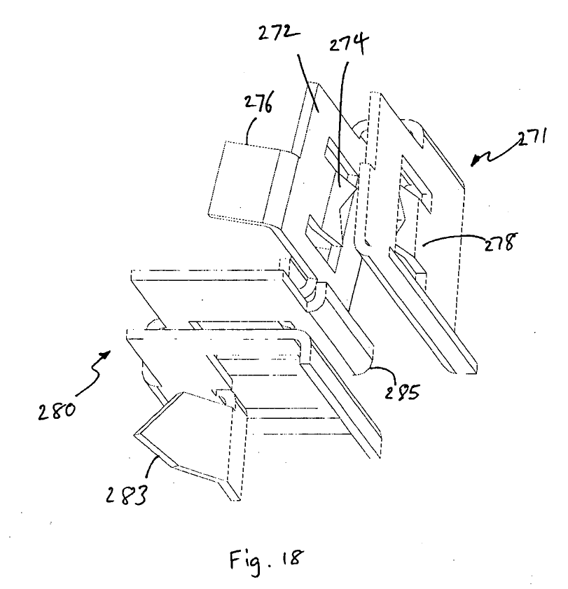

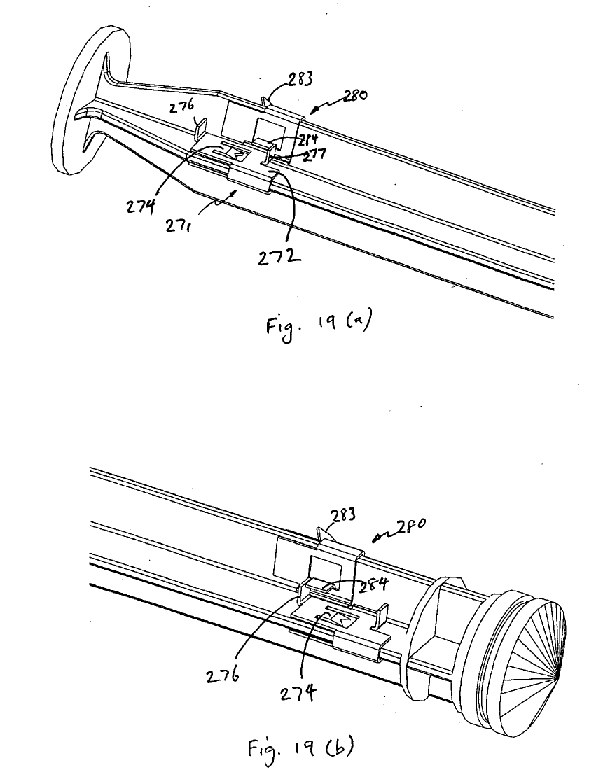

FIG. 14 is a perspective view of a lock release component of the ninth embodiment;

FIG. 15a is a perspective view of a syringe plunger with the components of FIGS. 12, 13 and 14 assembled around it, in its shipped state (not showing the syringe barrel);

FIG. 15b is a view similar to FIG. 15a showing only part of the plunger and showing the components with the lock release disengaged;

FIG. 15c is a view similar to FIG. 15b showing the components during expulsion of fluid from the syringe;

FIG. 15d is a view similar to FIG. 15b showing the components in a challenged state after drawing up and expulsion of less than a full syringe of liquid;

FIG. 15e is a perspective view of the components of FIGS. 12, 13 and 14 in a challenged state, with the syringe plunger and barrel omitted;

FIG. 16a is perspective view of a plunger lock component of a tenth embodiment of the invention;

FIG. 16b is a perspective view in a different direction of the component of FIG. 16a;

FIG. 17a is a perspective view of a barrel lock component of the ninth embodiment;

FIG. 17b is a perspective view from a different direction of the component of FIG. 17a;

FIG. 18 is a perspective view of the plunger lock and barrel lock components in an assembled state, omitting the syringe barrel and plunger;

FIG. 19a is a perspective view of part of a syringe plunger with the components of FIGS. 16, 17 and 18 assembled around it, in its shipped sate (not showing the syringe barrel); and

FIG. 19b is a view similar to FIG. 19a showing the components during expulsion of fluid from the syringe.

All the following embodiments are described principally with respect to a syringe having a nozzle, e.g. a luer connector, for attachment of a cannula, hypodermic or other needle or catheter line, etc. It will be appreciated that in every embodiment this nozzle could be replaced by a needle which is incorporated into the syringe at manufacture, e.g. moulded into the plastic of the barrel. It should also be understood that all of the following embodiments may be adapted to provide a frangible or weakened region on the plunger which is designed to break if excessive force is applied to the plunger. Alternatively the plunger may be made in more than one part which parts are designed to separate when excessive force is applied. In either case, the syringe is rendered inoperable or at least substantially inoperable.

Referring to FIGS. 1 and 2, a syringe 2 has a barrel 4 of transparent plastics material having an open end 6 having a widened rim 8 defining an indentation 10 of reduced diameter. The barrel 4 also has an outlet 12 having a needle (not shown) at the end thereof opposite from the open end 6 of the barrel 4.

A piston 14 is slidably received within barrel 4 and has a shaft 16 of plastics material extending from it and having a plunger handle 18 snap-fitted on the end thereof opposite to the piston 14. A safety bobbin or restrictor bobbin 20 of plastics material is slidably received within the barrel 4 and surrounds shaft 16. A gripping washer 22 surrounds the shaft 16 and has tines 24 of metal or plastics material harder than the plastic material of shaft 16 such that the washer 22 grips the shaft 16 and can slide in the direction of arrow A relative to the shaft 16 but cannot slide in the direction of arrow B (FIG. 1a) A second gripping member in the form of a plurality of tines 26 of metal or harder plastics material than the plastics material of inner wall of barrel 4 surrounds safety bobbin 20 such that tines 26 engage the inner wall of the barrel 4 in a manner such that the safety bobbin 20 can be moved relative to the barrel 4 in the direction of arrow A but cannot be moved in the direction of arrow B. The washer 22 can slide a limited distance d (FIG. 2) in either direction relative to the safety bobbin 20 between end walls 28, 30 of safety bobbin 20.

In order to assemble the syringe 2, the piston 14 together with the shaft 16, with the plunger handle 18 removed from the shaft 16, is inserted into the barrel 4 and pushed along the barrel until it abuts the end of the barrel adjacent to outlet 12. The safety bobbin 20, together with washer 22 and gripping member 26 is then placed around the shaft 16 and snap-fitted into the open end 6 of barrel 4. The safety bobbin 20 is prevented by indentation 10 and gripping member 26 from being removed from the barrel 4. The plunger handle 18 is then snap fit onto the end of shaft 16 remote from piston 14.

The operation of the syringe 2 will now be described.

The syringe 2 is provided by the manufacturer in sterile packaging (not shown) in the condition shown in FIG. 1a but with the plunger handle 18 mounted to the shaft 16. In order to fill the syringe 2, the needle (not shown) extending from outlet 12 is inserted into a reservoir of injectable liquid, or into the body of a patient, as a result of which liquid is drawn into the barrel 4, through outlet 12. The plunger handle 18 is then withdrawn in the direction of arrow B (FIG. 1a) to withdraw the piston 14 until it abuts the safety bobbin 20 as shown in FIG. 1b. In this position, the safety bobbin 20 is captured and locked in the barrel 4, and shaft 16 slides in the direction of arrow B relative to safety bobbin 20 and washer 22 until the piston 14 abuts end wall 30 of safety bobbin 20. At the same time, the washer abuts end wall 28 of safety bobbin 20.

In order to administer an injection, or expel bodily fluid such as blood from the syringe 2, the plunger handle 18 is then pushed in the direction of arrow A, as a result of which the piston 14 moves towards outlet 12 to expel liquid from the outlet 12. At the same time, because of the axial length of safety bobbin 20, rocking of the piston 14 and shaft 16 relative to safety bobbin 20 and barrel 4 is prevented. The washer 22 is prevented from moving in the direction of arrow B relative to shaft 16, but can move distance d relative to safety bobbin 20 until it abuts end wall 30 of safety bobbin 20. Thereafter, as the piston 14 moves in the direction of arrow A and barrel A, the safety bobbin 20 moves along barrel 4 until the piston 14 abuts the end wall of barrel 4 adjacent outlet 12 as shown in FIG. 1c.

If the piston handle 18 is at any point withdrawn in the direction of arrow B, the washer 22 can move distance d within safety bobbin 20 until piston 14 abuts end wall 30 of safety bobbin 20, or the washer 22 abuts the end wall 28 of safety bobbin 20. If the needle extending from outlet 12 has been inserted into a blood vessel, this action will withdraw blood into the barrel 4, which can then be seen by a user. It may also be possible to use this small degree of movement to agitate the drug in the syringe: this may be advantageous e.g. where a powdered drug needs to be agitated in order to ensure that it is fully dissolved into solution in a diluent contained in the syringe, prior to administration of the drug.

At the end of travel of the piston 14 and after the washer 22 abuts end wall 30 (or piston 14 abuts end wall 30) of safety bobbin 20, subsequent movement of the piston 14 in the direction of arrow B is limited only to the small float distance d, large functional movement is prevented by engagement of the gripping member 26 with the internal wall of barrel 4. If sufficient force is applied to the shaft 16 to overcome the resistance of gripping member 26, one or more, or even all, of the tines of gripping member 26 will damage the internal wall of barrel 4 of the syringe 2, resulting in an effective fluid seal between the piston 14 and the inner wall of the barrel 4 no longer being possible. In this way, withdrawal of piston 14 will no longer cause suction in barrel 4, as a result of which the syringe cannot be re-filled with injectable liquid and therefore cannot be re-used.

In a modification of the first embodiment shown in FIG. 3, the sliding washer 22 is replaced by a fixed washer 22a, whilst the sliding function is taken over by the gripping member which is slidably received on the exterior surface 20a of the bobbin 20.

It will be appreciated that the first embodiment, in either of its forms, could easily be adapted so that the direction in which the gripping member and washer resist motion is reversed. That is to say, the outer tines or barbs 26 would face distally so as to resist motion of the bobbin in the distal direction with respect to the barrel; the inner tines 24 would also face distally so as to resist motion of the plunger proximally with respect to the bobbin. In this event, the bobbin would be in a distal position in the device as manufactured. When the device is first used and the plunger is withdrawn by a user in order to draw up a fluid, the bobbin would be carried back with the plunger towards the proximal end of the syringe barrel. During the delivery stroke, i.e. movement of the plunger in a distal direction, the bobbin would remain fixed with respect to the barrel, and the plunger would move past the bobbin. This variation may also apply to one or more of the following embodiments.

A second embodiment is shown in FIG. 4. In this embodiment, the syringe consists of a barrel 30 with an open end through which the internal working components can be inserted. The other end is essentially closed, apart from a portal/nozzle 38 adapted for attachment of a needle or catheter as is conventional. The plunger includes a piston/bung or plunger head 34 and a circular cross section shaft. The shaft has two parts: a core 33 which is connected to the head 34 and to a handle 31, and an outer sleeve 32 which is slidable on the core 33.

The outer sleeve 32 is somewhat shorter in length than the plunger core 33. Movement of the sleeve 33 on the core 32 is limited to the distance D shown in FIG. 4c by engagement of the sleeve 32 with the plunger head 34 and the handle 31.

An annular restrictor bobbin 35, having a diameter smaller than the internal diameter of the syringe barrel, is located around the plunger sleeve 32. The central aperture of the restrictor bobbin 35 is slightly larger than the external diameter of the plunger sleeve 32. Affixed to both its outer periphery and to the wall of the central aperture are a number of tines or barbs 36, 37 respectively. The tines are directed in such a way that they will only permit movement of the plunger sleeve in the direction M shown in FIG. 4b.

During withdrawal of the plunger to draw up injectable or diluent, the outer tines 36 engage the interior wall of the syringe barrel and prevent the slidable restrictor bobbin from being displaced. A reduced diameter section 39 of the barrel at its extreme proximal end provides further security against the restrictor bobbin being either inadvertently or deliberately withdrawn from the barrel.

When the user depresses the plunger to expel the syringe contents, the plunger sleeve abuts the handle 31 and both outer shaft and core move distally. The iner tines 37 engage the plunger sleeve 32 and drag the restrictor bobbin 35 down the barrel of the syringe to a position at the extreme distl end of the syringe barrel. The outer tines 36 then prevent the subsequent withdrawal of the plunger to any substantial extent.

At any time during manual operation of the syringe, the plunger shaft core 33 and connected head 34 can be moved independently back and forth a small distance D. This allows for aspiration of a small quantity of blood or for repeated movment to assist in reconstituting a drug prior to injection.

Referring now to FIGS. 5a to 5d and to FIGS. 6a to 6d, a third embodiment is shown which operates on similar principles to the other embodiments but involves a syringe with a cruciform section rather than a circular one.

FIGS. 5a-5c show the third embodiment in (a) its position as supplied from the manufacturer, (b) the drawn back position and (c) the position after expulsion of the contents. These are largely self explanatory in view of the foregoing descriptions of the first and second embodiments.

FIG. 5d shows in somewhat more detail the restrictor bobbin of the third embodiment, which comprises a sector-shaped housing having external tines 52. The housing defines a recess in which is received a sliding element or member 57 which is provided with a further set of tines 54 constituting the internal tines of the overall bobbin. The distance over which repeated movement is provided by the bobbin is marked as D in FIG. 5d.

Further detail of this embodiment is shown in FIGS. 6a to 6d. FIG. 6a shows schematically a cross section of part of a syringe barrel 50 and plunger 51. Received in one quadrant of the cruciform plunger 51 is a slidable restrictor bobbin. As may be seen more clearly with reference to FIGS. 6b-d, the bobbin has a recess along running along the length of its corner edge which sits in the internal corner of the plunger shaft quadrant. Slidably received in this recess is a slider element 57 which is provided with barbs or tines 54 for engaging with the plunger shaft. A removable end cap 56 of the bobbin allows the slider element 57 to be inserted into the recess during manufacture. On the curved outer surface of the bobbin is a further tine or tines 52 which engage the barrel interior wall.

The operation of this embodiment will be easily understood from the description of previous embodiments. The slider element is captive within the bobbin and can move freely over the length of the recess, thus providing for a limited range of repeated movement for aspiration and/or assisting dissolution of powdered drugs. The direction of the respective tines 52, 54 will depend on where the bobbin is to be located in the manufactured syringe, as will be understood from the description of the previous embodiments.

FIGS. 7a and 7b show a fourth embodiment which is similar in most respects to the second embodiment shown in FIGS. 4a-c. The fourth embodiment relates to a syringe with a plunger having a cruciform section, where a slidable sleeve 62 is provided which fits into one quarter of the main cruciform plunger shaft core 63.

Double sets of inner and outer tines 67, 66 are shown; this arrangement may increase the stability of the bobbin 65. This double tine arrangement may be applied to any of the other embodiments for the same reasons.

FIGS. 8(a)-(d) show a fifth embodiment of the invention. As with previous embodiments, the syringe comprises a barrel 70 and plunger 71, the plunger shaft in this case being of circular cross section and having a stepped profile with regions 71a, 71b and 71c of different diameter. A restrictor bobbin 75 of generally U shaped cross section sits on the plunger shaft: a detailed view of the bobbin is provided in FIG. 8(d) which is an elevation of the bobbin alone in the direction A shown in FIG. 8(c).

The bobbin 75 is of similar design to that described in prior patent application number U.S. 2003/0060759, and the contents of this application are incorporated herein by reference. It is provided with a spring leaf 73 and outwardly and proximally oriented barbs 72. When installed in the syringe, the spring leaf bears resiliently against the interior wall of the syringe barrel 70, thereby urging the barbs 72 against an opposing portion of the barrel interior wall. At the same time, the spring leaf acting against the barrel wall urges the bobbin against the plunger 71. The bobbin is also provided with inwardly directed resilient tangs 74 which grip the plunger shaft, whilst allowing distal movement of the shaft with respect to the bobbin.

FIG. 8(a) shows the syringe in its starting position, with the plunger at its most distal position and the restrictor bobbin 75 located towards the proximal end of the barrel 70. The bobbin sits on the centre region 71b of the plunger shaft which is of smaller diameter than the most proximal region 71c but larger diameter than the most distal region 71a.

In use, a needle will be mounted on the luer connector nozzle 79 and an injectable liquid, e.g. a drug, will be drawn up into the syringe. In a modification of this embodiment, a syringe could be pre-fitted to the syringe in manufacture, in which case the luer connector in FIG. 8 would be replaced by a needle moulded into the plastic of the barrel 70.

Once the injectable has been drawn up, the plunger will be in the position shown in FIG. 8(b). Whilst the plunger is being drawn back, the bobbin remains stationary since any tendency for the plunger to carry the bobbin along with it would is been resisted by the barbs 72 engaging with the interior wall of the barrel. The bobbin now sits on the smallest diameter region 71a of the shaft, the tangs 74 having snapped inwardly against the smaller region 71a as they passed over the shoulder between the centre region 71b and the distal region 71a.

As can be seen in FIG. 8(b), the bobbin and plunger are free to move relative to one another over a limited distance defined by the clearance between the distal and proximal ends of the bobbin and the plunger head and plunger shoulder respectively. The length of the bobbin is selected so that enough movement is permitted to allow the aspiration of a small quantity or “flash” of blood as previously discussed. Note that it is not possible to retract the plunger further in the proximal direction so as to remove it and thereby remove the restrictor bobbin to allow further use of the syringe.

In FIG. 8(c) is shown the position of the plunger once the injectable has been delivered. The plunger is at the distal end of the barrel, having carried the restrictor bobbin with it: movement of the bobbin in the distal direction with respect to the barrel is of course permitted by the barbs 72. Once in this position, further retraction of the plunger is substantially prevented. Although the small degree of movement of the plunger permitted by relative sliding of plunger and bobbin is still possible, the dimensions of the components are chosen so that this degree of movement is insufficient to allow further injections.

A potential issue with the fifth embodiment is the possibility that the syringe may be used to draw up and deliver a relatively small volume of drug which did not require the plunger to be drawn back all the way to the position shown in FIG. 8(b). If the plunger is drawn back to a position intermediate those shown in FIGS. 8(a) and (b) then the bobbin may not snap into place in the distal region 71a of the plunger. In this case, the syringe could be used repeatedly. In a modification of this embodiment, a ratchet system is provided which operates between the bobbin and the central region 71b of the plunger. This could be provided simply by appropriate corrugations on the surface of the region 71b of the plunger, so that the tangs 74 of the bobbin engage with the corrugations to prevent distal movement of the plunger with respect to the bobbin. In this event, once any degree of proximal movement of the plunger has been made from the position shown in FIG. 8(a), subsequent distal movement will carry the bobbin with the plunger. This is the case until the bobbin snaps into the distal region of the plunger. Whilst in theory a second injection may still be possible, such a system would seriously impede attempts to use the syringe more than once. Corrugations on the central region 71b are shown in FIG. 8(c) only.

A sixth embodiment is shown in FIG. 9a. This embodiment is very similar to the first embodiment described in detail above with respect to FIGS. 1a-c and FIG. 2. Referring to FIG. 9a, a syringe barrel 81 containing a plunger having a shaft 86, is fitted with a restrictor bobbin 80. The outline of the restrictor bobbin 80 is identical with that of the first embodiment and the outer gripping member 87 is identical with that of the first embodiment. The proximal and distal directions in FIG. 9a are reversed from FIG. 2, so proximal is towards the left and distal is towards the right.

In this embodiment, the restrictor bobbin will start out in the syringe as supplied from the manufacturer in a distal position in the syringe barrel.

On drawing back the plunger (i.e. moving it to the left in FIG. 9a), the bobbin will be carried back with the plunger shaft 86 since the barbs 84 will be engaged with the plunger shaft. Once a desired quantity of fluid has been drawn up, the syringe needle (not shown) may be inserted into a patient and the plunger withdrawn to check for needle position by attempting to aspirate blood. If the restrictor bobbin has passed to the most proximal position possible for it (limited e.g. by a reduced diameter portion at the extreme proximal end of the syringe), then the free play allowed by the restrictor bobbin becomes important.

Unlike the first embodiment, the inner gripping washer 82 is fixed in the body of the restrictor bobbin 80 and is not slidable with respect to the body of the bobbin 80. Instead the washer 82 is fixedly mounted in the body of the restrictor bobbin 80. It is made of a springy material (stainless steel would be appropriate) and is so dimensioned that it is capable of resilient deformation as shown in dashed lines in FIG. 9a when a moderate pressure is applied to the plunger in the proximal direction C shown in FIG. 9a. It can easly be seen that the resilient deformation of the washer allows repeatable cycles of distal and proximal movement of the plunger with the barbs 84 of the washer 82 remaining in engagement with the plunger. The distance of the repeatable movement will be determined by the geometry of the washer and restrictor bobbin and, to some extent, the modulus of elasticity of the washer 82.

Referring now to FIG. 9b, a modification of the sixth embodiment is shown in which all parts are identical except the washer 82a which has a corrugated configuration. The washer is arranged to be in compression between the bobbin 80a and plunger shaft 86a. The corrugated configuration means that the washer is able more easily to be deformed, and the fact that it is in compression means that, as it deforms, it expands so that the barbs 84a are kept securely in engagement with the plunger 86a.

Referring now to FIG. 10a, a restrictor bobbin 95 of a seventh embodiment is shown, which is similar to the bobbin of the third embodiment (see FIG. 5d). The restrictor bobbin 95 has outward barns 92 for engaging with the barrel and inward barbs 94 for engaging with the plunger. The sliding element 57 of the third embodiment is replaced in the seventh embodiment with a resilient pad 97 e.g. of silicone rubber or other suitable strong elastomer which is securely fixed to the bobbin as shown. Mounted in the pad 97 are the inner barbs 94. In the seventh embodiment, a double set of barbs 94 is provided for increased stability.

Operation of the seventh embodiment will be apparent, but is shown for clarity in FIGS. 10b and 10c. In the extreme proximal position of the plunger 96, it may be desirable repeatedly to move the plunger through proximal and distal cycles of movement e.g. in order to attempt to aspirate blood from a patient. This is permitted by the resilient deformation of the pad 97. Arrows E and E′ in FIGS. 10b and 10c respectively show the direction of force on the plunger 96.

Referring now to FIGS. 1e-h, an eighth embodiment of the invention is a syringe which is pre-filled with diluent and is specifically for use with powdered/lyophilised drugs which require reconstitution by dissolution in a sterile solvent e.g. water.

A large proportion of injectable drugs, especially for use in developing countries, are supplied in powdered or lyophilised form in the vial. Prior to administration, a measured volume of sterile diluent is drawn up into a syringe, and the diluent then introduced into a vial of the drug, e.g. by passing the needle of the syringe through a pierceable septum on the vial. Although the introduction of the diluent, together with subsequent shaking of the vial, may be enough to cause the drug to pass completely into solution, it may also be helpful repeatedly to cycle some or all of the liquid between the syringe and vial.

This process presents two problems to the designer of a non-reusable syringe. The first is that if diluent is to be drawn up and injected into a vial using the same syringe as will be used to administer the drug—which is highly desirable—two complete cycles of proximal and distal movement of the plunger are necessary prior to the syring being rendered unusable. The second is that repeated cycling of the plunger is desirable to agitate the drug in the syringe and/or vial which is of course completely opposed to the objective of rendering the syringe unusable after a single operation.

A solution to the second of these issues is presented by a syringe which has a range of free repeatable movement which is sufficient to agitate the contents of a vial or to cycle a portion of it between syringe and vial, the range of free movement being sufficiently small that does not provide an opportunity for re-use of the syringe, or at least severely hampers re-use. Any of the previously described embodiments may be provided with a range of repeatable movement which is appropriate for this objective.

The first of the two problems still presents difficulty, however. It is in theory possible to use the small range of repeatable movement to transfer diluent to a vial of powdered drug in a number of small steps, but this may be undesirable because it would add to the total time needed to prepare and administer an injection. The eighth embodiment presents an alternative solution to the problem.

The syringe 102 shown in FIGS. 11a-e is supplied sterile packed in the state shown in FIG. 11a, filled with sterile water 100. The plunger comprises a head 114, circular cross section shaft core 116 and proximal end piece 115. Similar to the second embodiment, the shaft core 116 has received on it an outer shaft sleeve 117 which is slidable with respect to the core 116 over a limited distance determined by the difference in length of the sleeve 117 and core 116. The end piece 115, which will be described in more detail below, is a separate member which is installed on the end of the shaft core 116 during manufacture, after the sleeve 117 and other components have been installed on the shaft. This is not shown in the drawings.

Received onto the sleeve 117 is an annular restrictor bobbin 106 having a central bore and an outer diameter substantially the same as that of the internal diameter of the syringe barrel 104. The bobbin 106 is retained in the barrel by a flange 105 on the end of the barrel 104. The bobbin body is formed from plastics material, e.g. consists of a distal and a proximal moulding 106a, 106b respectively, between which is received a spring washer 122. The two mouldings 106a and 106b are secured together around the washer 122 during manufacture by adhesive, ultrasonic welding or any other suitable technique. When assembled, the bobbin body is an annular member having proximal and distal end faces 107, 108 and concentric annular inner and outer webs 109, 110. The webs 109, 110 each have four rectangular apertures 111 equally spaced around their circumference. In the proximal end face 107 are located four arc-shaped apertures 112.

Captive between the two halves 106a, 106b of the bobbin body is the washer 122. The washer 122 is of springy metal (stainless steel would be suitable) and comprises a ring like member having outer barbs 126 and inner barbs 124. These are best seen in FIG. 11h. The barbs protrude through respective apertures 111 in the webs 108, 109, whilst the main circular portion of the washer remains between the webs. Referring to FIG. 11a, in the device as manufactured the washer has a bowed profile such that both the inner and outer sets of barbs point distally.

The plunger end piece 115 comprises a unitary moulding of a suitable plastics material. It has a conventional end disc 118 at its proximal end and four arc shaped projections 119 extending distally from the end disc 118. The four projections 119 are in registry with the four apertures 112 in the proximal face 107 of the bobbin 106. Formations (not shown) on the plunger shaft 116, core 117 and bobbin 106 prevent relative rotation of these parts to ensure that the projections 119 remain in registry with the apertures 112 in the bobbin end face 107.

The first step in operating the syringe is shown in FIGS. 11b and 11c: the plunger is depressed to discharge the sterile water contents. The operator may choose how much of the water is discharged through a needle into a vial of powdered/lyophilised drug and how much is discarded. FIG. 11b shows the stage immediately prior to the plunger being fully depressed. The bobbin 106 has remained in its starting position in the barrel since the outer barbs 126 are oriented such as to resist distal movement of the bobbin with respect to the barrel 104. The projections 119 on the plunger end piece 105 have entered the apertures 109 in the proximal face 107 of the bobbin so that they touch the washer 122 at the annular apex of its bowed shape. When further pressure is applied to the plunger in the distal direction, the projections force the bowed shape of the washer 122 to flip into the opposite sense, as shown in FIG. 11c. The barbs on the projections now face proximally. It should be noted that because of the geometry of the bobbin body in relation to the washer, considerably more force would be required on either the inner or outer barbs to “flip” the washer than is required from the projections 119.

The sequence of operation shown in FIGS. 11d and 11e is similar to that of previous embodiments: one full withdrawal of the plunger followed by one full depression of the plunger is permitted, with a degree of free movement provided by the plunger sleeve 117 to allow aspiration of blood or agitation of the contents of a vial.

A ninth emodiment will now be described with reference to FIGS. 12 to 15. In this embodiment, an additional safety feature is incorporated, namely the prevention of any re-use of the syringe even if the first use does not involve full retraction of the plunger. A drawback with the embodiments described above is that in most cases it is possible to use e.g. a 5 ml syringe to draw up e.g. 2.5 ml and then to expel the liquid leaving the restrictor element half way down the usable extent of the syringe. A further retraction of the plunger to draw up 2.5 ml of liquid and subsequent injection of that liquid is then possible.

The additional safety feature of the 9th embodiment is provided by means of a mechanism which triggers after the first distal movement of the plunger, such that movement of the plunger in the proximal direction is restricted.

The 9th embodiment is based on a standard 5 ml syringe with a cruciform section plunger shaft. Fitted between the plunger shaft and the barrel, as with the other embodiments, is a restrictor element. In this embodiment, the restrictor element comprises two pressed stainless steel components 201, 220 termed the plunger lock and the barrel lock and shown, respectively, in FIGS. 12 and 13. An additional component 240, known as the lock release element, is shown in FIG. 14.

Referring firstly to FIG. 12, the plunger lock 201 comprises a unitary body of pressed stainless steel formed into a main arch 202 with first and second side walls 203, 204. The first side wall 203 is the wall whose external face is visible in FIGS. 12(c) and (d) whilst the second side wall 204 is the wall whose external face is visible in FIG. 12(a).

The terms “top” and “bottom” as used with reference to this embodiment refer to the top and bottom of the component as it is shown in FIG. 12. The terms “proximal” and “distal” will be used to refer to the right hand end and left hand end, respectively, of the component as it is shown in FIG. 12(a), which equates to the left hand end and right hand end, respectively, as shown in FIG. 12(c).

As best seen in FIG. 12(c), the first side wall 203 of the main arch 202 is formed with an inwardly and proximally directed tang 206 whose free end is formed with twin points 207. This feature will be termed the distal barb, because it acts to prevent distal movement of the plunger with respect to the restrictor element (the restrictor element in this embodiment being constituted by the plunger lock of FIG. 12 and the barrel lock of FIG. 13).

The second side wall 204 of the main arch 202 (as best seen in FIG. 12(a)) is formed with a flap 208 which extends downwardly and outwardly of the second side wall 204 and which then curves back inwardly towards the side wall 204. The free end of the flap 208 is formed as a barb 209 which is positioned so as to engage with the plunger surface when the component is installed on the plunger, and is oriented so as to oppose proximal movement of the plunger. The barb 209 is termed the proximal barb, because it acts to prevent proximal movement of the plunger with respect to the restrictor element. The proximal barb 209 is best seen in FIG. 12(d).

On the proximal edge of the flap 208 is an inwardly directed flange 212. At the bottom of the second side wall 204 is a laterally and proximally extending tang 210, whose proximal end is formed as an upwardly turned flange 211. The function of these features will be explained later.

Turning now to FIG. 13, the barrel lock 220 is shown in three views corresponding to FIGS. 12(a), 12(b) and 12(c). Since the views correspond, the terms top, bottom, distal and proximal will be used in the same way, i.e in FIG. 13(a) the proximal end is on the right of the figure and the distal end is on the left. It should be noted, however, that the plunger component and barrel component are not both aligned in the same way when they are assembled together as a restrictor bobbin in a syringe (See e.g. FIG. 15).

The barrel lock 220, like the plunger lock, is formed with a main arch 222 having a first side wall 223 and a second side wall 224. Extending laterally from the bottom of each side wall 223, 224 are first and second plates 225, 226 respectively. The proximal outer tip of the second plate 226 is formed as a proximally oriented barb 227 which, when the component is installed in a syringe, bears against the inner wall of the syringe barrel to resist proximal movement of the barrel lock component 220 with respect to the syringe barrel. The outer edge of the first plate 225 has a protrusion 228 with rounded corners which is adapted, in use, to bear against the opposing side of the syringe barrel interior wall, to provide a reaction force for the barb 227.

The proximal end of the first plate 225 is turned downwards at approximately 245 degrees to the plane of the plate 225. The feature is called the lock cinch 229, and its function will be explained later.

Turning now to FIG. 14, a lock release component 240 is shown. This component does not form part of the movable restrictor bobbin which is composed of the plunger lock 201 and the barrel lock 220. The function of the lock release is to “latch” the proximal barb 209 of the plunger lock 201 out of contact with the plunger until an appropriate point in the sequence of steps for using the syringe, i.e. until it is desired to prevent further substantial proximal movement of the plunger.

The lock release component 240 comprises a main arch 242 with first and second side walls 243, 244 respectively. At the bottom of each side wall 243, 244, first and second plates 245, 246 extend laterally. At the outer end of each plate 245, 246 is a respective end region 247, 248 respectively, which is angled slightly upwards out of the plane of the plates 245, 246. At the outer end of each end region are twin barbs 249 facing in opposite directions which, when the component is installed in a syringe, engage with the internal wall of the syringe barrel to secure the component against any movement with respect to the barrel. On the proximal side of each of the plates 245, 246 are upwardly turned flanges 250, 251 respectively. There is also an outwardly turned flange 252 on the proximal end of the second side wall 244.

Projecting distally from the distal end of the second side wall 244 is a latch tang 253 whose function is to hold the proximal barb 29 of the plunger lock out of engagement with the plunger, as will be explained in more detail below.

The assembled state of the various components of the ninth embodiment, and its operation, will now be described with reference to FIG. 15 which shows the components assembled on a syringe plunger. The syringe barrel has been omitted for clarity, but its form and location and its interaction with the illustrated components will be readily understood by anyone of ordinary skill in this art.

Referring firstly to FIG. 15(a) a syringe plunger assembly 260 comprises a plunger shaft 261 of generally cruciform section, that is to say in its transverse section will have the shape of a cross with four limbs at 90 degrees to each other. The plunger shaft is fitted with an elastomeric plunger or plunger head 262 at its distal end. At its proximal end, the plunger shaft terminates in a disc shaped plate 263 for a user to manipulate the plunger assembly 260. All these components are conventional.

The components of FIGS. 12, 13 and 14 may be seen in their assembled state in FIG. 15. The details are best seen in FIG. 15(b) which shows the components somewhat separated. The plunger lock 201 is located with its main arch 202 extending around a first limb 261a of the plunger shaft 261, and with its tang 210 extending between the surface of a second limb 261b of the plunger shaft 261 and the first side wall 223 of the barrel lock component 220. The plunger lock does not engage with the internal barrel wall.

The barrel lock 220 has its main arch 222 extending over the second limb 261b of the plunger, which is at 90 degrees to the limb 261a over which the plunger lock 21 is mounted. Although the barrel of the syringe is not shown, it will be appreciated that the outer edge 228 of the barrel lock and the barrel lock barb 227, which is hidden behind the plunger in FIG. 15, will bear against opposing regions of the inner surface of the barrel wall. The barrel lock has no other barb and is thus freely movable with respect to the plunger, unless its movement is limted by engagement with another component of the device e.g. the plunger lock 201.

The lock release 240 is disposed with its main arch 242 extending around the first limb 261a of the plunger shaft 261. Opposed barbs 249 of the lock release may be seen clearly in FIG. 15(b); these are engaged with the barrel in a manner which will be understood readily by someone of ordinary skill in this art, such that the lock realease 240 is immovably retained with respect to the barrel. There are no other barb elements on the lock release, and thus the plunger shaft and lock release are freely movable with respect to each other.

The device is shown in its shipped state in FIG. 15(a). In this figure, the plunger should be assumed to be fully or substantially fully depressed into the barrel, i.e. substantially at its most extreme distal position. The three pressed steel components 201, 220 and 240 are all located in the region of the proximal end of the barrel and plunger. The latch tang 253 of the lock release 240 extends between the first limb 261a of the plunger shaft and the flap 28, flange 212 and proximal barb 209 of the plunger lock 201. The first side wall 223 of the barrel lock 220 extends over the tang 210 of the plunger lock and the first plate 225 of the barrel lock extends over the first side wall 204 and flap 208 of the plunger lock 201.

The proximal barb 209 of the plunger lock is biased towards the first limb 261a of the plunger shaft by virtue of the geometry of the main arch 202 and the resilience of the stainless steel of which it is made. At the same time the distal barb 206 of the plunger lock (which may be seen in FIG. 15(e)) is biased towards the opposite side of the first limb 261a of the plunger shaft. Whilst the distal barb 206 engages with the plunger shaft to resist distal movement of plunger with respect to the plunger lock, the latch tang 253 of the lock release 240 comes between the proximal barb 209 and the plunger and prevents the proximal barb 209 from engaging with the plunger shaft. The proximal barb 209 may slide relatively freely over the latch tang 253 since the latch tang 253 is made of stainless steel rather than the polypropylene of the plunger shaft.

When the syringe is used, the first step is to pull back the plunger, normally either to draw up a liquid medicament or to draw up a blood sample from a patient. As the plunger is drawn back (i.e. moved in the proximal direction), the shaft moves freely past the lock release 240 and barrel lock 220. The distal barb 206 of the plunger lock is biased against the plunger shaft, but the barb is oriented so as not to create substantial resistance to proximal movement of the plunger. The plunger lock 201 is prevented from moving proximally by engagement of the flap 208 and flange 212 on the plunger lock with the lock cinch 229 of the barrel lock 220. The interaction of the cinch 229 with the flange 212 to prevent proximal movement of the plunger lock 201 with respect to the barrel lock 220 is shown more clearly in FIG. 15(e). Thus the components remain essentially in their shipped state as shown in FIG. 15(a) whislt the plunger is retracted either fully or partially.

The plunger of the syringe is then depressed (i.e. moved distally) to expel the contents which have been drawn up. The state of the components after an initial small movement of the plunger in the distal direction is shown in FIG. 15(b). As the plunger moves distally, the plunger lock 201 moves with it since the distal barb 206 acts to prevent relative movement of the plunger lock and plunger shaft in this sense. Since the lock release 240 is fixed to the barrel, the proximal barb 209 of the plunger lock 201 slides off the latch tang 253 as the plunger moves distally. FIG. 15(b) shows the proximal barb disengaged from the latch tang 253 and bearing against the first limb 261a of the plunger shaft.

The barrel lock 220 remains in its shipped state until, as the plunger lock 201 moves distally, the flange 211 at the proximal end of the tang 210 of the plunger lock 201 engages with the proximal edge of the first side wall 223 of the barrel lock 220, as shown in FIG. 15(b). At this point the barrel lock 220 is drawn along with the plunger lock 201; the barb 227 of the plunger lock is engaged with the interior wall of the barrel (not shown) but the orientation of the barb 227 is such that distal movement of the barrel lock in the barrel is not resisted.

FIG. 15(c) shows a state when the plunger has been depressed further to expel the contents of the syringe.

FIG. 15(d) shows the state of the components if a user attempts to draw back the plunger from the state shown in FIG. 15(c). As the plunger is drawn back, the plunger lock is forced to move back with it because the proximal barb 209 on the plunger lock acts to prevent proximal movement of the the plunger relative to the plunger lock. The plunger and plunger lock move proximally until the flange 212 on the plunger lock encounters the lock cinch 229 on the barrel lock. At this point, no further proximal movement is possible because the barrel lock barb 227 prevents proximal movement of the barrel lock with respect to the plunger.

Additional security is provided in this design by the design of the cinch 229 and flange 212. These components are angled such that increased force on the plunger to withdraw it will cause increased force on the barb 209 in the direction of the surface of the first plunger limb 261a, thereby increasing the resistance of the plunger lock against proximal movement.

It will be appreciated that the barrel lock 220 and plunger lock 201 allow unlimited repeated movement of the plunger over a distance which is limited at one end by the engagement of the cinch 229 and flange 212 (FIGS. 15(d) and 15(c)), and at the other end by engagement of the flange 211 with the proximal edge of the first side wall 223 of the barrel lock (see FIGS. 15(b) and 15(c)). As discussed in relation to the other embodiments, this movement allows for withdrawal of a flash of blood prior to administering an injection to check the position of the needle in a patient.

A tenth embodiment will now be described with reference to FIGS. 16 to 19. Similar to the ninth embodiment, this embodiment incorporates the additional safety feature of preventing re-use of the syringe even if the first use does not involve completely filling the syringe. The tenth embodiment also incorporates the “lost motion” feature, as with all the other embodiments, which allows unrestricted free movement of the plunger over a limited distance. The tenth embodiment differs from the ninth in that these objectives are achieved with only two components in addition to the standard syringe components.

FIG. 16 shows a plunger lock component 271 of pressed stainless steel. The proximal end of the component is to the right in FIG. 16(a). The terms “top” and “bottom” regarding this component will be used for this component as it is represented in FIG. 16(a). The component comprises a general U shape with first and second side walls 272, 273. In the first side wall 272 is an aperture from which projects a barb 274 which is oriented inwardly so as to resist proximal movement of the plunger with respect to the plunger lock 271; this will be referred to as the proximal barb 274. At each end of the first wall 272 are proximal and distal flanges 276, 277 respectively. In the second side wall 273 (see FIG. 16(b)) is a further aperture from which projects a further barb 278 which is oriented inwardly so as to resist distal movement of the plunger with respect to the plunger lock 71; this will be referred to as the distal barb 78.

FIG. 17 shows a plunger lock 280 also comprising a generally U shaped member of pressed stainless steel having first and second side walls 281, 282 respectively. The proximal end of the plunger lock 280 is the right hand end as it is represented in FIG. 17(a). The terms “top” and “bottom” will be used as they relate to the component as it is depicted in FIG. 17(a), though it should be bourne in mind that, as with the ninth embodiment, the plunger lock and barrel lock are not oriented in the same sense when installed in a syringe (see FIGS. 18 and 19).

Projecting laterally and proximally from the first side wall 281 of the barrel lock, and inclined upwardly, is a barb 283 oriented to resist movement of the barrel lock in a proximal direction; this is referred to as the proximal barb 283 on the barrel lock. On the second side wall 282 of the barrel lock is a laterally extending limiting flange 284. The base of the second wall 282 is turned outwardly to form a latch flange 285. The function of these flanges will be explained later.

FIG. 18 shows the plunger and barrel locks in their shipped state with the plunger and barrel hidden. The components are shown in the same state in FIG. 19(a) with the plunger shown; in FIG. 18 the components have been represented in an orientation which shows the latch flange 285 of the barrel lock 280 engaged with the first side wall 272 of the plunger lock 271 so as to hold the proximal barb 274 out of contact with the plunger.

The function of the various components will be apparent from the drawings. In the shipped state, shown in FIG. 19(a) and FIG. 18, the proximal barb 274 does not prevent proximal movement of the plunger which can therefore be retracted to draw up fluid. Initial distal movement of the plunger, as with the ninth embodiment, causes the latch 285 to be released; as the plunger moves distally it carries the plunger lock 271 with it and the first side wall 272 of the plunger lock slips off the latch 285, allowing the proximal barb 274 to engage the plunger. Thereafter, further movement of the plunger in the distal direction causes the proximal flange on the plunger element to engage with the limiting flange 284 of the barrel lock thereby dragging the barrel lock with it. This state is shown in FIG. 19(b).

As with the ninth embodiment, once the latch 285 is disengaged, proximal movement of the plunger becomes impossible, apart from the limited free movement allowed by the relative movement between the plunger and barrel locks (limited by the proximal and distal flanges 276, 277 on the plunger lock engaging with the limiting flange 284 on the barrel lock). This is because the barb 283 on the barrel lock prevents proximal movement of the barrel lock, the plunger lock cannot move proximally with respect to the barrel lock because of interaction between the flanges 284 and 276, and the plunger cannot move proximally past the plunger lock because of the proximal barb 274.

Modified forms of the ninth and tenth embodiments are contemplated where the barrel lock component is provided with a distal barb or the like in addition to its proximal barb, and the plunger lock is provided only with its distal barb. In these modified embodiments, a latch member would be provided to prevent engagement of the distal barb of the barrel lock until the plunger has been drawn back and then expulsion of fluid has commenced. The latch could be provided e.g. as a projection on the plunger lock or alternatively on a separate member as in the ninth embodiment. In the latter case, the separate member would be mounted on the plunger, most appropriately on or adjacent the proximal side of the plunger head.

In these modified versions of the ninth and tenth embodiments, the plunger and barrel locks would be at the distal end of the syringe in the shipped state. Retraction of the plunger would move both components proximally up the syringe, within the plunger. Distal movement of the plunger would then un-latch the distal barb of the barrel lock, which would then be immobilised with respect to the barrel.

A potential issue with the ninth and tenth embodiments is that there may be a small jerk when the latch is disengaged as the plunger is initially depressed. As previously stated, this could potentially cause discomfort to a patient if the needle has been inserted into the patient at this point. However, it is a simple matter to make the initial movment of the plunger prior to insertion of the needle into the patient, and in fact this is normal practice in order to expel any air in the syringe prior to administering an injection.

1. A a syringe comprising:
(a) a plunger including a plunger head and a shaft;
(b) a barrel;
(c) a restrictor bobbin located between the shaft and the barrel and having an outer barb, tine, serration or the like engaged with the barrel and oriented so as to limit movement of the bobbin with respect to the barrel in a first direction;
(d) the restrictor bobbin and/or shaft carrying a formation for limiting movement of the bobbin with respect to the plunger in the said first direction;
(e) the bobbin being provided with a further barb, tine, serration or other formation oriented or arranged so as to restrict movement of the bobbin with respect to one of the barrel and plunger shaft in a second direction opposed to the first direction;
(f) a latch member for disabling the said restricting function of the further barb, tine, serration or other formation until a predetermined point in an operation cycle of the syringe.

2. A syringe as claimed in claim 1 wherein the said predetermined point in the operation cycle is an initial movement of the plunger in a distal direction.

3. A syringe as claimed in claim 1 wherein the barrel has a cylindrical interior surface substantially free of discontinuities between proximal and distal ends of a full range of usable movement of the plunger head in the barrel.

4. A syringe as claimed in claim 1 wherein the said formation carried by the restrictor bobbin and/or shaft for limiting movement in the first direction comprises a barb, tine, serration or the like which is engaged with the plunger shaft surface.

5. A syringe as claimed in claim 1 wherein the the plunger and bobbin are freely slidable with respect to each other over a limited range of movement, or the plunger or bobbin have relatively movable parts, whereby unlimited repeated proximal and distal cycles of plunger head movement with respect to the barrel are permitted over a predetermined limited range which is less than the said full usable range of movement of the plunger head.

6. A device as claims in claim 1 wherein the said latch member comprises a separate member secured to the barrel.

7. A device as claims in claim 6 wherein the said latch member comprises a projection which, in an engaged state, extends distally between the plunger shaft and the said further barb, tine, serration or the like on the restrictor bobbin.

8. A device as claimed in claim 5 wherein the bobbin comprises two relatively movable parts or the bobbin is deformable such that first and second regions of the bobbin are capable of changing their relative disposition, and wherein the said latch member comprises a projection extending from one of the said parts or regions of the bobbin.

9. A device as claimed in claim 7 wherein the said projection extends between the plunger or barrel surface and the said further barb, tine, serration or other formation on the other of the said two parts of the bobbin.

10. As syringe as claimed in claim 1 wherein the said latch member comprises a separate member secured on or adjacent the proximal side of the plunger head, which in an engaged state extends between the barrel interior wall and a portion of the restrictor bobbin.
